(19) United States
(12) Patent Application Publication
(10) Pub. No.: US 2013/0338778 A1
(43) Pub. Date: Dec. 19, 2013

(54) SPINAL DISC ANNULUS CLOSURE DEVICE

(71) Applicants: Hagay Drori, Tel Aviv (IL); Hamid Sharim, Kokhav Yair (IL); Roey Shafrir, Modiin (IL); Gil Naor, Hofit (IL)

(72) Inventors: Hagay Drori, Tel Aviv (IL); Hamid Sharim, Kokhav Yair (IL); Roey Shafrir, Modiin (IL); Gil Naor, Hofit (IL)

(73) Assignee: NEWVERT LTD., Netanya (IL)

(21) Appl. No.: 14/002,717

(22) PCT Filed: Mar. 7, 2012
(86) PCT No.: PCT/IL12/50071

§ 371 Date: Sep. 1, 2013

Related U.S. Application Data
(60) Provisional application No. 61/450,638, filed on Mar. 9, 2011.
(60) Provisional application No. 61/534,911, filed on Sep. 15, 2011.

(51) Int. Cl. A61F 2/44

(52) U.S. Cl. CPC A61F 2/442
USPC 623/17.16

(57) ABSTRACT

An implant (10) arranged as a tubular member (20) with a longitudinal axis (80). The tubular member exhibits a proximal end (100) and a distal end (110), the distal end arranged to extend to within the inner region of an annulus. Inter-annulus support members (30) are arranged radially about the tubular member, and connected proximally of the distal end, thus defining an axial support member (120) between the connection to the tubular member and the distal end. Proximal securing members are further provided arranged radially about the tubular member and removed proximally from the connection point of the inter-annulus support members. The inter-annulus support members are connected to the axial support member such that ejection forces are opposed by a concavingly curved link member between the axial support member and the inter-annulus support members.

### CROSS-REFERENCE TO RELATED APPLICATIONS

This application claims priority from U.S. Provisional Patent Application Ser. No. 61/450,638, filed Mar. 21, 2011 and U.S. Provisional Patent Application Ser. No. 61/534,911, filed Sep. 28, 2011, both entitled “SPINAL DISC ANNULUS CLOSURE DEVICE”, the entire contents of each of which are incorporated herein by reference.

### FIELD OF THE INVENTION

The invention relates generally to the field of implantable devices for the repair and closure of a spinal annular defect, and more particularly to an implant arranged to securely seal an annular defect with improved anti-ejection characteristics.

### BACKGROUND OF THE INVENTION

The human spine, known technically as the vertebral column, is constituted of a plurality of articulating vertebrae, and extending downwards towards fused vertebrae in the sacrum and coccyx. Using standard anatomical terminology, the vertebral column is found in the dorsal aspect of the torso. The articulating vertebrae are separated from adjacent vertebrae on either side by an invertebral disc which forms a cartilaginous joint to allow slight movement of the vertebrae, and further acts to hold the various vertebrae together so as to form the vertebral column.

Each invertebral disc comprises an outer annulus fibrosus, often simply called the annulus, which surrounds and contains the nucleus pulposus which is a jelly-like substance which functions to distribute hydraulic pressure within each invertebral disc under compressive loads. In the event of an invertebral disc defect, such as a prolapsed or herniated disc, the nucleus pulposus is forced out through the defect of the annulus, and may apply pressure to nearby nerves or to the spinal cord. In severe cases the escaping nucleus pulposus may cause chemical irritation of nearby nerve roots. Protrusion of the nucleus pulposus may be variously referred to as a disc bulge, a herniated disc, a ruptured disc or a sequestered disc, depending on the specific diagnosis.

While various schemes for repair of the annulus defects are known, one common solution is a surgical procedure known as discectomy which involves the surgical removal of the herniated disc material. Discectomy is often performed in conjunction with a laminectomy, where a small piece of bone, known as the lamina, is removed from the affected vertebra, allowing the surgeon to better see and access the area of disc herniation.

One problem with the above procedure is that additional nucleus pulposus material may be ejected from the annulus over time by the unsealed defect in the annulus, which is not sealed by the discectomy. Thus, a device and associated procedure is required to seal the annulus defect. Various devices and procedures are known to the prior art, including without limitation, WIPO Patent Publication S/N WO 2010/089717 entitled “Implantable Device for Sealing a Spinal Annular Fissure Tear and Method for Deploying the Same”, the entire contents of which are incorporated herein by reference. One issue not fully addressed by the above subject patent publication, and other devices of the prior art, is the issue of ejection, i.e. the tendency of any device placed in the annulus to be ejected over time responsive to forces developed in the remaining nucleus pulposus material.

In order to avoid confusion in describing medical devices, certain fixed terminology is utilized. In particular, the term proximal usually means closer to the surgeon, unless otherwise stated, and the word distal usually means further removed from the surgeon, unless otherwise stated. Surgery to repair a defect in the annulus is usually performed from the patient's dorsal side, i.e. from the back, and thus the terms proximal and distal are understood with the surgeon approaching from the patient's back; however this is not meant to be limiting in any way. In the event of surgery performed ventrally, the terms need to be understood in relation to a dorsal operation.

What is desired, and not supplied by the prior art, is a device arranged to seal the annulus against further release of nucleus pulposus material through the defect, which is designed to itself resist ejection from the annulus.

### SUMMARY

Accordingly, it is a principal object of the present invention to overcome at least some of the disadvantages of the prior art. In certain embodiments this is provided by an implant arranged as a tubular member with a longitudinal axis. The tubular member exhibits a proximal end and a distal end, the distal end arranged to extend to within the inner region of the annulus. Inter-annulus support members are arranged radially about the tubular member, and connected proximally of the distal end, thus defining a axial support member between the connection to the tubular member and the distal end. Ejection forces arrayed against the tubular member are reflected by the tubular member and resisted by the tubular member body in cooperation with the inter-annulus support members. Such an arrangement advantageously converts a portion of the ejection forces to forces in line with the longitudinal axis of the tubular member thus increasing the resistance to ejection, as the constituent material of the tubular member resists buckling.

Proximal securing members are further provided arranged radially about the tubular member and removed proximally from the connection point of the inter-annulus support members. In one embodiment an extension of the proximal securing members, when deployed, are arranged to meet the outer surface of the annulus in which the implant has been implanted, while an extension of the inter-annulus support members, when deployed, are arranged to meet the inner surface of the annulus in which the implant has been implanted. In another embodiment proximal securing members, when deployed, are arranged to meet the medial wall of the annulus in which the implant has been implanted, i.e. the channel formed in the annulus by the discectomy, while the inter-annulus support members, when deployed, are arranged to meet the inner surface of the annulus in which the implant has been implanted. There is no requirement that the distance between the inter-annulus support members and the proximal securing members be fixed, and an arrangement provided for a variable distance is disclosed herein.

The implant may further provide flow blocking members arranged to prevent the flow of nucleus pulposus through the central portion of the tubular member. The implant may further provide a barrier material, arranged to cooperate with the tubular member so as to prevent the release of nucleus pulposus, particularly by blocking the flow of nucleus pulposus external of the outer portion of the tubular member. In one particular embodiment, the barrier material may be arranged to further act as a scaffold for stem cells or other biological products. In another embodiment, the barrier material is replaced with material arranged to serve as a scaffold for stem cells or other biological products.

In one embodiment the implant is delivered to the implant site in a restrained condition within a delivery device, and upon release from the delivery device the inter-annulus support members urge to a deployed configuration in the absence of the restrainment. Similarly, upon release from the delivery device the proximal securing members urge to a deployed configuration in the absence of the restrainment. In another embodiment the inter-annulus support members and/or the proximal securing members are constituted of a shape memory alloy, and deployment is responsive to body heat.

In one independent embodiment, an implant for repair of a spinal inter-vertebral disc is provided, the implant comprising: an axial support member exhibiting a longitudinal axis, an outer surface, a distal end and a proximal end; and at least one inter-annulus support member having a first end, a second end opposing the first end, a first link member, a first face and a second face opposing the first face, the first link member arranged to connect the first end of the at least one inter-annulus support member to the axial support member, the at least one inter-annulus support member having a deployed configuration wherein the second end of the at least one inter-annulus support member extends away from the longitudinal axis of the axial support member, the first face of the first link member generally facing the axial support member in the deployed configuration and generally concavingly curved, and the second face of the first link member generally convexingly curved, wherein the first face and second face of the first link member generally proceed proximally from the axial support member along an extension axis into the curve, the extension axis exhibiting an acute angle with the longitudinal axis, the acute angle defined from the second face of the first link member to the longitudinal axis.

In one embodiment, the implant further comprises: at least one proximal securing member positioned proximal of the at least one inter-annulus support member, the at least one proximal securing member having a first end and a second end opposing the first end, the first end of the at least one proximal securing member connected to the axial support member. In one further embodiment, the at least one proximal securing member comprises a plurality of proximal securing members arranged radially about the axial support member.

In another further embodiment, the at least one proximal securing member has a delivery configuration wherein the at least one proximal securing member does not extend past a plane defined by the outer surface of the axial support member, and a deployed configuration wherein the second end of the at least one proximal securing member extends away from the longitudinal axis of the axial support member past the plane defined by the outer surface of the axial support member. In one yet further embodiment, the at least one proximal securing member, in the deployed configuration, urges to expand.

In one yet even further embodiment, the implant is arranged to be deployed within a tear in the annulus of the spinal inter-vertebral disc and the distance between the second of the at least one plurality of proximal securing members and the longitudinal axis of the axial support member, when the at least one proximal securing member is in an at rest state, is 0.5-3 millimeters greater than the radius of the tear. In one yet even further embodiment, the distance between the second end of the at least one proximal securing member and the longitudinal axis of the axial support member, when the at least one proximal securing member is in the at rest state, is about 1.5 millimeters greater than the radius of the tear.

In one further embodiment, the at least one proximal securing member is displaced from the at least one inter-annulus support member along the longitudinal axis of the axial support member by the thickness of a target annulus, such that the second face of the at least one inter-annulus support members meets the inner wall of the target annulus and the at least one proximal securing member meets the outer wall of the target annulus. In another further embodiment, the at least one proximal securing member is displaced from the at least one inter-annulus support member along the longitudinal axis of the axial support member by less than the thickness of a target annulus, such that the second face of the at least one inter-annulus support member meets the inner wall of the target annulus and the at least one proximal securing member meets the medial portion of the target annulus.

In one further embodiment, the second end of the at least one proximal securing member is wider than the first end of the at least one proximal securing member. In another further embodiment, the at least one proximal securing member is constituted of wires.

In one further embodiment, the plurality of inter-annulus support members, the plurality of proximal securing members and the axial support member are formed of a unitary tube. In another further embodiment, the at least one proximal securing member is displaced from the at least one inter-annulus support member along the longitudinal axis of the axial support member by an adjustable length.

In one further embodiment, one of the axial support member, the at least one inter-annulus support member and the at least one proximal securing member is porous. In another further embodiment, the implant further comprises a spring arranged to connect the at least one proximal securing member to the axial support member.

In one further embodiment, the at least one proximal securing member comprises a plurality of layers. In one yet further embodiment, the distance between adjacent layers of the plurality of layers is 0-4 millimeters.

In one yet even further embodiment, the distance between adjacent layers of the plurality of stacked layers is about 0.3 millimeters. In another yet further embodiment, each of the plurality of stacked layers exhibits a thickness of 0.05-0.5 millimeters.

In one yet even further embodiment, each of the plurality of stacked layers exhibits a thickness of about 0.25 millimeters. In one further embodiment, the at least one proximal securing member comprises a plurality of proximal securing members, and wherein each of the plurality of proximal securing members extends into a plane, the plane of a first proximal securing member exhibiting an angle with the longitudinal axis of the axial support member different than an angle of the plane of a second proximal securing member.

In another embodiment, the at least one inter-annulus support member comprises a plurality of inter-annulus support members. In one embodiment, the at least one inter-annulus securing member exhibits a delivery configuration wherein the at least one inter-annulus support member does not extend past a plane defined by the outer surface of the axial support member.

In one further embodiment, the at least one inter-annulus support member moves from the delivery configuration to the deployed configuration responsive to body heat. In another further embodiment, the at least one inter-annulus support member moves from the delivery configuration to the deployed configuration responsive to release from a restraining device, the at least one inter-annulus support member held in the delivery configuration within the restraining device.

In one embodiment, the second end of the at least one inter-annulus support member is wider than the first end of the at least one inter-annulus support member. In another embodiment, the at least one inter-annulus support member comprises a pair of first link members, the pair of first link members exhibiting an angle of 45°-90° between each other.

In one embodiment, the at least one inter-annulus support member is constituted of a wire. In another embodiment, the implant further comprises a barrier material arranged to block the flow of nucleus pulposus from an annulus of the spinal inter-vertebral disc to external of the axial support member.

In one further embodiment, the barrier material is one of a braided sheet, a polymer skin and a nano-fiber web. In another further embodiment, the barrier material comprises a first face, a second face opposing the first face and at least one slit arranged to mate with the at least one inter-annulus support member, the first face of the at least one inter-annulus support member facing the first face of the barrier material at the first link member and the second face of the at least one inter-annulus support member facing the second face of the barrier material at the second end of the at least one inter-annulus support member.

In one embodiment, the implant further comprises a first flow blocking member having a first end and a second end opposing the first end, the first end of the first flow blocking member connected to the axial support member, the second end of the first flow blocking member extending towards the longitudinal axis of the axial support member when the at least one inter-annulus support member is in the deployed configuration. In another embodiment, the axial support member is solid.

In one embodiment, each of the inter-annulus support members comprises a plurality of layers. In one further embodiment, the distance between adjacent layers of the plurality of layers is 0-4 millimeters.

In one yet further embodiment, the distance between adjacent layers of the plurality of stacked layers is about 0.3 millimeters. In another further embodiment, each of the plurality of stacked layers exhibits a thickness of 0.05-0.5 millimeters.

In one yet further embodiment, each of the plurality of stacked layers exhibits a thickness of about 0.25 millimeters. In another embodiment, the at least one inter-annulus support member, in the deployed configuration, extends into a plane, the plane exhibiting an angle with the longitudinal axis of the axial support member of 45-120 degrees, wherein the angle is defined between the first face of the at least one inter-annulus support member and the longitudinal axis of the axial support member.

In one embodiment, the at least one inter-annulus support member, in the deployed configuration, extends into a plane, the plane exhibiting an angle with the longitudinal axis of the axial support member of less than, or equal to, 90 degrees, wherein the angle is defined between the first face of the at least one inter-annulus support member and the longitudinal axis of the axial support member. In one further embodiment, the plane exhibits an angle with the longitudinal axis of the axial support member of 45-90 degrees, wherein the angle is defined between the first face of the at least one inter-annulus support member and the longitudinal axis of the axial support member.

In another embodiment, the implant is arranged to be deployed within a tear in the annulus of the spinal inter-vertebral disc and the at least one inter-annulus support member, in the deployed configuration, meets the inner wall of the target annulus at a distance of at least 1 millimeter from an edge of the tear. In one further embodiment, the at least one inter-annulus support member, in the deployed configuration, meets the inner wall of the target annulus at a distance of 1-12 millimeters away from an edge of the tear.

In one embodiment, the implant further comprises: a proximal securing member positioned proximal of the at least one inter-annulus support member, the at least one proximal securing member having a first end and a second end opposing the first end, the first end of the at least one proximal securing member connected to the axial support member, wherein the at least one inter-annulus support member generally extends along a support member axis and the proximal securing member generally extends along a securing member axis, the support member axis rotated about the longitudinal axis of the axial support member in relation to the securing member axis. In another embodiment, the at least one inter-annulus support member comprises a shape memory polymer.

In one embodiment, the implant further comprises: a proximal securing member positioned proximal of the at least one inter-annulus support member, the at least one proximal securing member having a first end and a second end opposing the first end, the first end of the at least one proximal securing member connected to the axial support member, wherein the axial support member, the at least one inter-annulus support member and the proximal securing member each comprise a shape memory polymer. In another embodiment, the distance between the first and second end of at least two of the plurality of inter-annulus support members are different.

In one embodiment, the axial support member comprises an elastic material. In another embodiment, the implant further comprises: a lateral inter-annulus support member having: a first end; a second end opposing the first end; a second link member; an arm; a first face; and a second face opposing the first face, wherein the second link member is arranged to connect the first end of the lateral inter-annulus support member to the axial support member, wherein the lateral inter-annulus support member has a delivery configuration wherein the lateral inter-annulus support member does not extend past a plane defined by the outer surface of the axial support member, and a deployed configuration wherein the second end of the lateral inter-annulus support member extends away from the longitudinal axis of the axial support member past the plane defined by the outer surface of the axial support member, wherein the first face of the second link member generally faces the axial support member in the deployed configuration and generally convexingly curves from the second end of the axial support member and the second face of the second link member generally concavingly curves from the second end of the axial support member, the first face and second face of the second link member generally proceeding along an extension axis exhibiting an acute angle with the longitudinal axis into the curve, the acute angle being defined from the first face of the second link member, wherein the convex curve of the first face of the second link member extends into the first face of the arm, the first face of the arm generally concavingly curved from the first face of the second link member, and wherein the concave curve of the second face of the second link member extends into the second face of the arm, the second face of the arm generally convexingly curved from the second face of the second link member. In one further embodiment, the lateral inter-annulus support member comprises a plurality of layers.

In one embodiment, the implant further comprises a second flow blocking member connected to the distal end of the axial support member, the second flow blocking member arranged to prevent the extrusion of regenerative material contained within the axial support member. In another embodiment, the at least one inter-annulus support member comprises a plurality of layers, each layer arranged in the deployed configuration to arrest movement of an adjacent layer, at a predetermined point, caused by force applied to the adjacent layer.

In one further embodiment, each layer of the at least one inter-annulus support member exhibits a hole arranged to receive an end of an adjacent layer. In another further embodiment, each layer of the at least one inter-annulus support member exhibits a protrusion arranged, in the deployed configuration, to come in contact with an end of an adjacent layer, thereby arresting movement of the adjacent layer, at a predetermined point, caused by force applied to the adjacent layer.

In one embodiment, each inter-annulus support member, in the deployed configuration, extends into a plane, the plane of a first inter-annulus support member exhibiting an angle with the longitudinal axis of the axial support member different than an angle of the plane of a second inter-annulus support member. In another embodiment, the axial support member exhibits a distance of greater than 1 mm between the first end and the second end of the axial support member.

In one independent embodiment, a method for repairing a spinal inter-vertebral disc is provided, the method comprising: providing an implant comprising: an axial support member exhibiting a longitudinal axis, an outer surface, a distal end and a proximal end; and at least one inter-annulus support member having a first end, a second end opposing the first end, a first link member, a first face and a second face opposing the first face, the first link member arranged to connect the first end of the at least one inter-annulus support member to the axial support member, the at least one inter-annulus support member having a deployed configuration wherein the second end of the at least one plurality of inter-annulus support members extends away from the longitudinal axis of the axial support member, the first face of the first link member generally facing the axial support member in the deployed configuration and generally concavingly curved, and the second face of the first link member generally convexingly curved, wherein the first face and second face of the first link member generally proceed proximally from the axial support member along an extension axis into the curve, exhibiting an acute angle with the longitudinal axis, the acute angle defined from the second face of the first link member to the longitudinal axis; delivering the provided implant into a target annulus; and moving the at least one inter-annulus support member into the deployed configuration, wherein in the deployed configuration the at least one inter-annulus support member is arranged to come in contact with an inner wall of the target annulus.

In one embodiment, the method further comprises: providing a flow blocking member connected to the distal end of the axial support member; and depositing regenerative material within the axial support member, wherein the provided flow blocking member is arranged to prevent the extrusion of the deposited regenerative material from the axial support member.

In another embodiment, the method further comprises: providing a lateral inter-annulus support member having: a first end; a second end opposing the first end; a second link member; an arm; a first face; and a second face opposing the first face, wherein the second link member is arranged to connect the first end of the lateral inter-annulus support member to the axial support member, wherein the lateral inter-annulus support member has a delivery configuration wherein the lateral inter-annulus support member does not extend past a plane defined by the outer surface of the axial support member, and a deployed configuration wherein the second end of the lateral inter-annulus support member extends away from the longitudinal axis of the axial support member past the plane defined by the outer surface of the axial support member, wherein the first face of the second link member generally faces the axial support member in the deployed configuration and generally convexingly curves from the second end of the axial support member and the second face of the second link member generally concavingly curves from the second end of the axial support member, the first face and second face of the second link member generally proceeding along an extension axis exhibiting an acute angle with the longitudinal axis into the curve, the acute angle being defined from the first face of the second link member, wherein the convex curve of the first face of the second link member extends into the first face of the arm, the first face of the arm generally concavingly curved from the first face of the second link member, and wherein the concave curve of the second face of the second link member extends into the second face of the arm, the second face of the arm generally convexingly curved from the second face of the second link member; and moving the provided lateral inter-annulus support member into the deployed configuration such that the provided lateral inter-annulus support member is juxtaposed with a tear in the posterior wall of the target annulus, wherein the delivering is through a lateral wall of the target annulus.

In one embodiment, the method further comprises: inserting a nucleus pulposus prosthesis into the intervertebral disc of the target annulus, wherein the delivered implant is arranged to secure the inserted nucleus pulposus prosthesis.

Additional features and advantages of the invention will become apparent from the following drawings and description.

### BRIEF DESCRIPTION OF THE DRAWINGS

For a better understanding of various embodiments of the invention and to show how the same may be carried into effect, reference will now be made, purely by way of example, to the accompanying drawings in which like numerals designate corresponding elements or sections throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIGS. 1A-1B. illustrate various views of an implant for repair of a spinal inter-vertebral disc in a deployed configuration, comprising a plurality of inter-annulus support members, a plurality of proximal securing members and a axial support member, according to certain embodiments;

FIG. 1C illustrates a high level schematic diagram of the implant of FIGS. 1A-1B inserted in the spine wherein the proximal securing members are displaced from the plurality of inter-annulus support members by less than the thickness of a target annulus;

FIG. 1D illustrates a high level schematic diagram of the implant of FIGS. 1A-1B inserted in the spine, wherein the proximal securing members are displaced from the plurality of inter-annulus support members by at least the thickness of the target annulus;

FIG. 1E illustrates a high level schematic diagram of a bottom view of the implant of FIGS. 1A-1B inserted in the spine;

FIGS. 2A-2C illustrate various views of an implant for repair of a spinal inter-vertebral disc in a deployed configuration, further comprising a plurality of flow blocking members, according to certain embodiments;

FIGS. 3A-3B illustrate various views of an implant for repair of a spinal inter-vertebral disc in a deployed configuration, further comprising a barrier material;

FIGS. 3C-3D illustrate various constitutions of the barrier material of FIGS. 3A-3B;

FIGS. 4A-4B illustrate various side views of the implant of FIGS. 2A-2C in a delivery configuration;

FIG. 5A illustrates a perspective view of a delivery system for delivering an implant into a target annulus;

FIGS. 5B-5D illustrate various stages in the deployment of an implant into the target annulus;

FIG. 6 illustrates a perspective view of a non-limiting embodiment of the production process of the implant of FIGS. 2A-2C;

FIGS. 7A-7G illustrate various views and positions of an implant for repair of a spinal inter-vertebral disc, wherein the displacement of proximal securing members from the plurality of inter-annulus support members is adjustable;

FIGS. 8A-8B illustrate various views of an implant for repair of a spinal inter-vertebral disc, comprising a plurality of inter-annulus support members, a plurality of proximal securing members and a axial support member, wherein the plurality of inter-annulus support members and the plurality of proximal support members are constituted of wires;

FIGS. 9A-9D illustrate various views of an implant for repair of a spinal inter-vertebral disc in a deployed configuration, comprising a plurality of inter-annulus support members, a plurality of proximal securing members and a axial support member, wherein the plurality of inter-annulus support members and the plurality of proximal support members each comprise a plurality of layers;

FIG. 9E illustrates a high level schematic diagram of a side cut view of the implant of FIGS. 9A-9D inserted in a target annulus, specifically illustrating the plurality of inter-annulus support members;

FIG. 9F illustrates a high level schematic diagram of a bottom view of the implant of FIGS. 9A-9D inserted in a target annulus;

FIG. 10 illustrates a perspective view of an implant for repair of a spinal inter-vertebral disc in a deployed configuration, comprising a plurality of inter-annulus support members, a plurality of proximal securing members and a axial support member, wherein the plurality of inter-annulus support members each comprise a plurality of layers; and

FIG. 11 illustrates a high level flow chart of a first method for repairing a spinal inter-vertebral disc;

FIG. 12A illustrates a high level side view of an implant for repair of a spinal inter-vertebral disc in a deployed configuration, the implant comprising a plurality of inter-annulus support members, a plurality of proximal securing members and a axial support member, wherein the plurality of inter-annulus support members exhibit differing lengths;

FIG. 12B illustrates a high level schematic view of the implant of FIG. 12A inserted in a spine;

FIG. 13A illustrates a high level side view of a portion of an implant for repair of a spinal inter-vertebral disc in a deployed configuration, the implant comprising a plurality of inter-annulus support members, a plurality of proximal securing members, a axial support member and a lateral inter-annulus support member;

FIG. 13B illustrates a high level schematic view of the implant of FIG. 13B inserted in a spine;

FIG. 13C illustrates a high level flow chart of a method of operation of the implant of FIGS. 13A-13B;

FIGS. 14A-14B illustrate various views of an implant for repair of a spinal inter-vertebral disc in a deployed configuration, the implant comprising a plurality of inter-annulus support members, a plurality of proximal securing members and a axial support member, wherein the plurality of inter-annulus support members each comprise a plurality of layers, each layer exhibiting holes arranged to receive the ends of an adjacent layer;

FIGS. 15A-15B illustrate various views of an implant for repair of a spinal inter-vertebral disc in a deployed configuration, the implant comprising a plurality of inter-annulus support members, a plurality of proximal securing members and a axial support member, wherein the plurality of inter-annulus support members each comprise a plurality of layers, each layer exhibiting a protrusion arranged to block the movement of an adjacent layer;

FIG. 16 illustrates a high level perspective view of an implant for repair of a spinal inter-vertebral disc in a deployed configuration, the implant comprising a plurality of inter-annulus support members, a plurality of proximal securing members and a axial support member, wherein the axial support member and the plurality of proximal securing members are connected by a spring;

FIGS. 17A-17B illustrate various views of an implant for repair of a spinal inter-vertebral disc in a deployed configuration, the implant comprising a plurality of inter-annulus support members, a plurality of proximal securing members and a axial support member, wherein each inter-annulus support member and each proximal securing member is arranged to bend independently; and

FIG. 18 illustrates a high level flow chart of a second method for repairing a spinal inter-vertebral disc.

### DETAILED DESCRIPTION

Before explaining at least one embodiment in detail, it is to be understood that the invention is not limited in its application to the details of construction and the arrangement of the components set forth in the following description or illustrated in the drawings. The invention is applicable to other embodiments being practiced or carried out in various ways. Also, it is to be understood that the phraseology and terminology employed herein is for the purpose of description and should not be regarded as limiting.

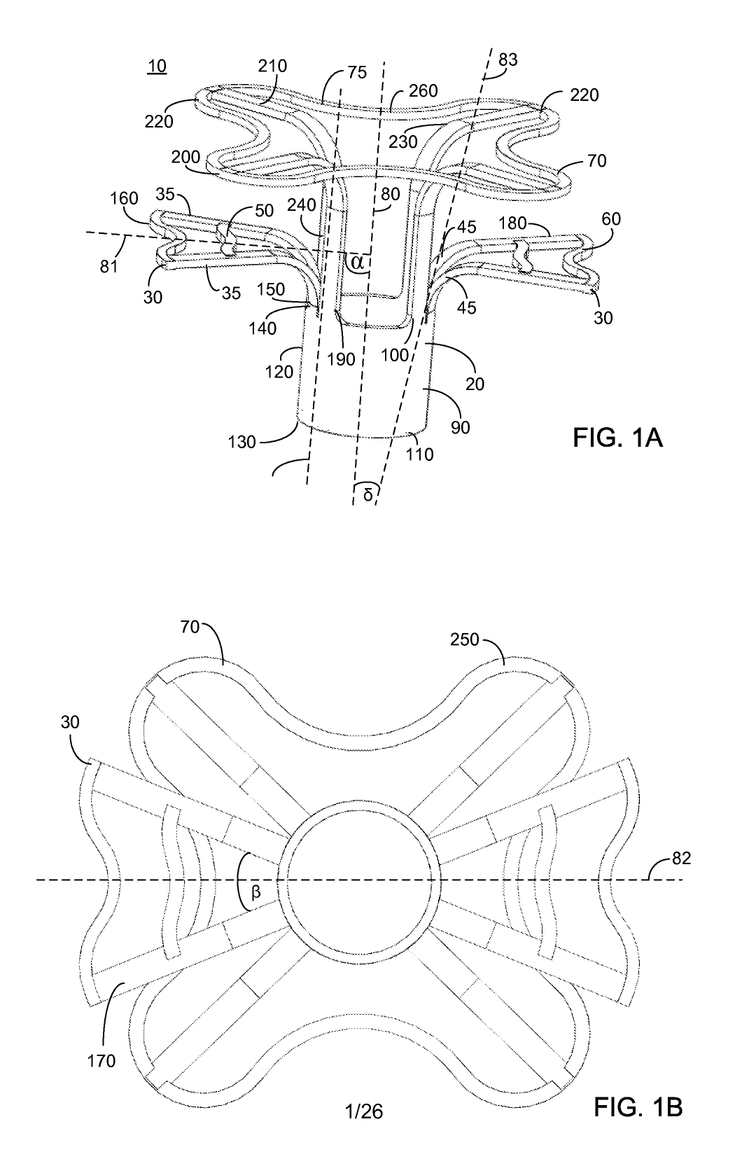

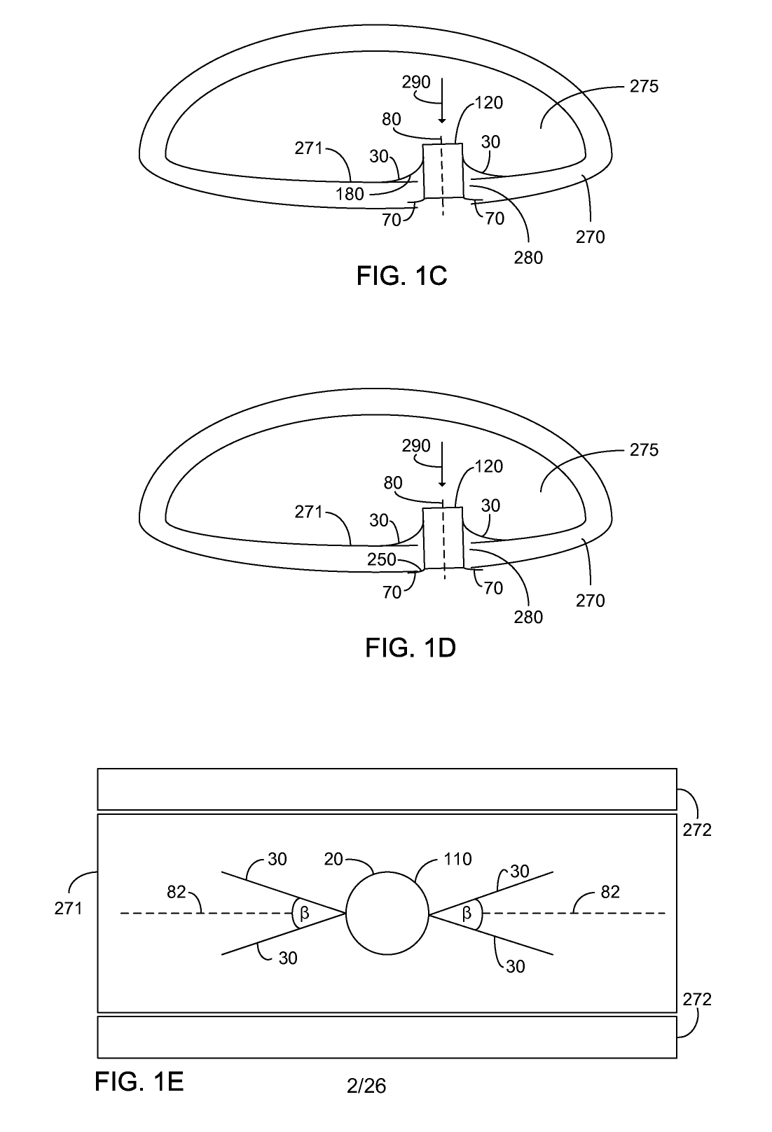

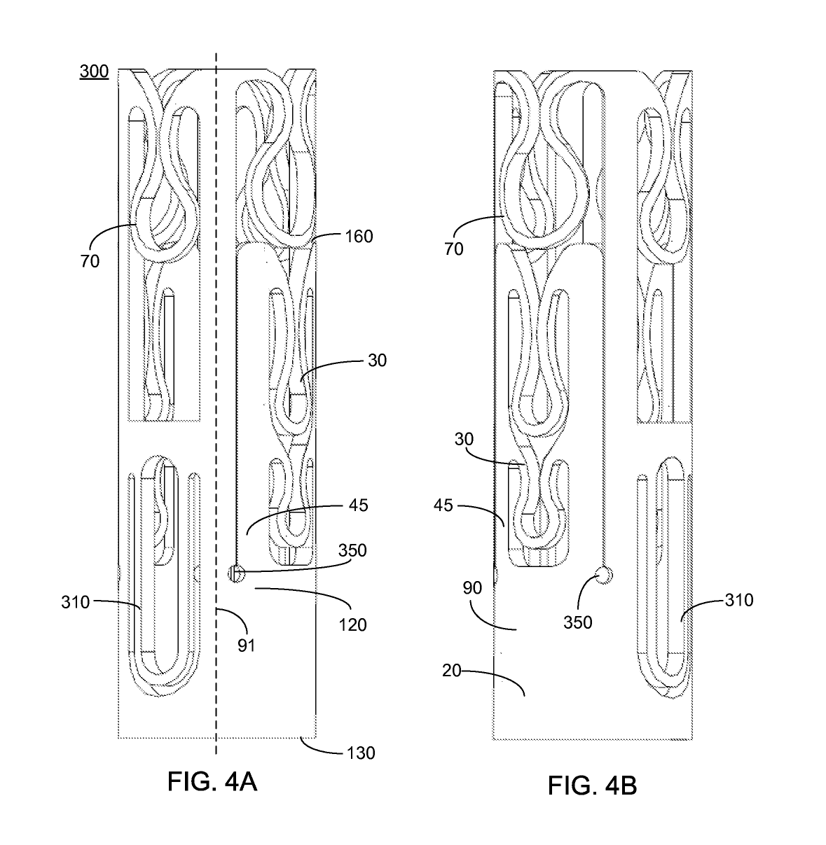

FIG. 1A illustrates a perspective view of an implant 10 for repair of a spinal inter-vertebral disc in a deployed configuration; FIG. 1B illustrates a view from a distal end of implant 10 in the deployed configuration; FIG. 1C illustrates a high level schematic view of implant 10 inserted in a spine wherein proximal securing members are displaced from a plurality of inter-annulus support members by less than the thickness of a target annulus 270; FIG. 1D illustrates a high level schematic diagram of implant 10 inserted in the spine wherein the proximal securing members are displaced from the plurality of inter-annulus support members by at least the thickness of target annulus 270; and FIG. 1E illustrates a high level schematic diagram of a bottom view of implant 10 inserted in the spine, the figures being described together. Implant 10 comprises: a generally tubular member 20; a plurality of inter-annulus support members 30 and a plurality of proximal securing members 70. In one embodiment, generally tubular member 20, plurality of inter-annulus support members 30 and plurality of proximal securing members 70 each comprise a polymeric material. In one embodiment, plurality of inter-annulus support members 30 and plurality of proximal securing members 70 each exhibit superelastic properties. In one embodiment, as will be described below in relation to FIGS. 4A-4B, plurality of inter-annulus support members 30 and plurality of proximal securing members 70 are each constituted of a shape memory alloy. In one further embodiment, plurality of inter-annulus support members 30 and plurality of proximal securing members 70 each comprise Nitinol. In one embodiment, generally tubular member 20 comprises an elastic material. In one embodiment, one or more of generally tubular member 20, inter-annulus support members 30 and proximal securing members 70 comprise a shape memory polymer.

Each of the inter-annulus support members 30 comprises a pair of arms 35, a pair of link members 45, an inter-annulus inner connecting member 50, an inter-annulus outer connecting member 60 and exhibits: a first end 150; a second end 160, opposing first end 150; a first face 170; and a second face 180, opposing first face 170. Each proximal securing member 70 comprises an arm 210, an outer link member 220, an inner link member 230 and a proximal extender 240, is connected to adjacent proximal securing members 70 by a proximal connecting member 75 and exhibits a first end 190, a second end 200 opposing first end 190, a first face 250 and a second face 260 opposing first face 250.

Generally tubular member 20 exhibits: a longitudinal axis 80 extending through the center of generally tubular member 20; an outer surface 90 defining a plane 91; a proximal end 100; a distal end 110; and a axial support member 120, formed by a distal portion of generally tubular member 20 and exhibiting a first end 130 defining distal end 110 of generally tubular member 20 and a second end 140 facing proximal end 100 of generally tubular member 20. In one non-limiting embodiment, generally tubular member 20 exhibits a generally circular cross section and axial support member 120 in one further embodiment comprises a hollow inner portion, thus forming an annulus. In another non-limiting embodiment (not shown), generally tubular member 20 exhibits a generally elliptic cross section. In another non-limiting embodiment (not shown), generally tubular member 20 exhibits a generally oval cross section. In another non-limiting embodiment (not shown), generally tubular member 20 exhibits a generally rectangular cross section. In one embodiment, axial support member 120 comprises a hollow inner portion. In another embodiment, axial support member 120 is solid.

First end 150 of each inter-annulus support member 30 is consonant with outer surface 90, and in particular is in a line with outer surface 90 parallel to longitudinal axis 80 and defines second end 140 of axial support member 120, and each inter-annulus support member 30 extends radially away from first end 150 towards second end 160 in a plane 81. As will be described below, in operation implant 10 is delivered through a tear 280 of annulus 270 and inter-annulus support members 30 are arranged to meet inner wall 271 of annulus 270. In one embodiment, an angle α defined between plane 81 and longitudinal axis 80, i.e. from plane 81 defined by first face 170 of second end 160 of each inter-annulus support member 30 to longitudinal axis 80 is 45°-90°. Advantageously, second end 160 of inter-annulus support member 30 does not meet inner wall 271, thereby when inter-annulus support member 30 presses against inner wall 271 pressure is applied by the curved portion of link members 45 or the generally flat portion of arms 35 and not the edge of second end 160, which could damage inner wall 271. In one embodiment, arms 35 and link members 45 are each constructed and arranged such that the contact point of each inter-annulus support member 30 with inner wall 271 is 1-12 mm from the edge of tear 280 and in one further embodiment is about 6 mm from the edge of tear 280. In one embodiment, inter-annulus support members 30 extend generally along a support member axis 82.

First end 190 of each proximal securing member 70 is consonant with outer surface 90, and in particular is in a line with outer surface 90 parallel to longitudinal axis 80, and each proximal securing member 70 extends radially away from first end 190 towards second end 200. Each proximal extender 240 extends proximally from second end 140 of axial support member 120 in a line generally parallel with longitudinal axis 80, in the plane defined by outer surface 90, to the respective inner link member 230. Inner link member 230 exhibits a curve extending into a first end of a respective arm 210, and each arm 210 extends in a plane, in one embodiment generally orthogonal to longitudinal axis 80 and proximal of inter-annulus support members 30. A second end of each arm 210, opposing the first end thereof, is connected to a respective outer link member 220. First face 250 of each proximal securing member 70 faces second face 180 of one of the plurality of inter-annulus support members 30, and second face 260 of each proximal securing member 70 faces the patient dorsal direction. In one embodiment inner link members 230 each comprise an elastic material, and inner link members 230 and arms 210 are each constructed and arranged such that in an at rest condition the distance between second end 200 of each proximal securing member 70 and longitudinal axis 80 is 0.5-3 mm greater than the radius of tear 280 of annulus 270, preferably about 1.5 mm greater than the radius of tear 280.

In one embodiment, second end 160 of each inter-annulus support member 30 is wider than first end 150 thereof. In particular, an angle β is defined between link members 45 of each inter-annulus support member 30. In one embodiment angle β is 45°-90°, in one particular embodiment angle β is about 80°. In one embodiment, second end 200 of each proximal securing member 70 is wider than first end 190 thereof. In one embodiment, the distance between first end 130 and second end 140 of axial support member 120 is greater than 1 mm.

Plurality of inter-annulus support members 30 are arranged radially about generally tubular member 20. Two inter-annulus support members 30 are illustrated, however this is not meant to be limiting in any way, and more than two inter-annulus support members 30 may be provided without exceeding the scope. Each of pair of arms 35 is connected to second end 140 of axial support member 120 by a respective link member 45. Each of plurality of inter-annulus inner connecting members 50 and plurality of inter-annulus outer connecting members 60 are arranged to connect a pair of arms 35 and provide support thereto. Each inter-annulus outer connecting member 60 is arranged to connect a pair of arms 35 at an end thereof consonant with second end 160. Plurality of proximal securing members 70 are arranged radially about generally tubular member 20 proximal of plurality of inter-annulus support members 30 and first end 190 of each proximal securing member 70 is connected to a proximal extension of generally tubular member 20. Four proximal securing members 70 are illustrated, however this is not meant to be limiting in any way, and fewer, or more, proximal securing members may be provided without exceeding the scope.

As indicated above, second end 160 of each inter-annulus support member 30 extends away from longitudinal axis 80 of generally tubular member 20 and extends past plane 91 defined by outer surface 90 of generally tubular member 20. In one embodiment, first face 170 of each inter-annulus support member 30 faces axial support member 120 and concavingly curves away from second end 140 of axial support member 120 to plane 81 defined by arms 35, and in one embodiment second face 180 of each inter-annulus support member 30 convexingly curves away from second end 140 of axial support member 120 to plane 81. In particular, in one embodiment link member 45 is connected to axial support member 120 defining second end 140 thereof, first face 170 of link member 45 generally proceeds from second end 140 of axial support member 120 along longitudinal axis 80 into the concave curve and second face 180 of link member 45 generally proceeds from second end 140 of axial support member 120 along longitudinal axis 80 into the convex curve. In another embodiment, link member 45 generally proceeds from second end 140 of axial support member 120 along an axis 83 into the curve. An acute angle δ is defined between axis 83 and longitudinal axis 80, acute angle δ being defined from second face 180 of link member 45 to longitudinal axis 80. Thus, inter-annulus support member 30 is at all times preferably proximal of a straight line drawn from first end 150 to second end 160 of the respective inter-annulus support member 30.

In one embodiment, link members 45 and axial support member 120 are formed as a unitary body, thus ensuring maximum resistance to ejection forces applied thereto. In an exemplary embodiment, plurality of inter-annulus support members 30 are formed as part of generally tubular member 20. In one embodiment, plurality of proximal securing members 70 are formed as part of generally tubular member 20. In one embodiment, as illustrated in FIG. 1C, plurality of proximal securing members 70 are displaced from plurality of inter-annulus support members 30 along longitudinal axis 80 by less than the thickness of annulus 270. In another embodiment, as illustrated in FIG. 1D, plurality of proximal securing members 70 are displaced from plurality of inter-annulus support members 30 along longitudinal axis 80 by at least the thickness of annulus 270.

As illustrated in FIG. 1C, axial support member 120 and plurality of inter-annulus support members 30 are situated within the enclosed area of annulus 270, i.e. in an area 275 comprising the nucleus pulposus. At least a portion of second face 180 of each inter-annulus support member 30 meets inner wall 271 of annulus 270. Annulus 270 exhibits a tear 280, optionally comprising a surgically created channel created as part of the discectomy, and implant 10 is inserted through tear 280 into area 275 of annulus 270. The portion of generally tubular member 20 extending along longitudinal axis 80 proximal of plurality of inter-annulus support members 30 and plurality of proximal securing members 70 are situated within tear 280. Advantageously, the construction of inter-annulus support members 30 and proximal securing members 70 is porous thereby scar tissue grows into and around inter-annulus support members 30 and proximal securing members 70, thus affixing implant 10 to annulus 270 over time.

When an ejection force 290 is applied from area 275 to implant 10, implant 10 is urged to move proximally through tear 280. Plurality of inter-annulus support members 30 pressed against inner wall 271 of annulus 270 oppose the ejection forces. Additionally and advantageously, ejection force 290 is generally parallel to longitudinal axis 80 and thus the shape of first face 170 of link members 45, which as described above is in one embodiment concave, and the connection to axial support member 120 significantly increases the resistance to ejection force 290 as compared to the purely elastic resistance of prior art systems, since any significant bending of link members 45 responsive to ejection force 290 would result in buckling at the connection point between axial support member 120 and link members 45. Advantageously, the arrangement of implant 10 thus resists ejection force 290 responsive to both buckling and bending resistance, which represents an increased resistance in relation to exclusively bending resistance of the prior art.

In one embodiment, as described above, second face 180 of each inter-annulus support member 30 comes in contact with inner wall 271 of annulus 270 at a distance of 1-12 mm from the edge of tear 280. Advantageously, the tissue in the portion of inner wall 271 of annulus 270 displaced from tear 280 is healthier and can withstand greater pressure than the degenerated tissue of the area surrounding tear 280 which may further tear from the pressure applied by inter-annulus support members 30. Additionally, as described above, in one embodiment inter-annulus support members 30 extend generally along support member axis 82. Preferably, implant 10 is positioned such that support member axis 82 is generally parallel to the vertebrae 272 adjacent annulus 270, as illustrated in FIG. 1E. Angle β between each adjacent pair of link members 45 causes the points of contact of each inter-annulus support member 30 with inner wall 271 to be displaced from support member axis 82 in the superior and inferior directions, i.e. in the directions of adjacent vertebrae 272. This is advantageous since a tear 280 is usually formed at the portion where annulus 270 is thinnest. Thus, distancing the point of contact between inter-annulus support members 30 and inner wall 271 in the superior and inferior directions presents inter-annulus support members 30 with a thicker portion of annulus 270 thereby reducing the risk of further damage to annulus 270. Furthermore, due to the shape of inter-annulus support members 30, which as described above is in one embodiment concave, responsive to ejection force 290 applied to implant 10, inter-annulus support members 30 extend further along inner wall 271 of annulus 270 thereby further distancing themselves from tear 280 and advancing towards vertebrae 272, thereby applying pressure to a more healthier and thicker portion of annulus 270. Plurality of inter-annulus inner connecting members 50 and plurality of inter-annulus outer connecting members 60 are arranged to prevent unnecessary movement of the respective arms 35 of inter-annulus support members 30 within annulus 270.

Friction between proximal securing members 70 and the inner wall of tear 280 prevents movement of implant 10, particularly responsive to forces applied thereto in directions which differ from the direction of ejection force 290. In one embodiment, in the deployed configuration proximal securing members 70 are not at rest but instead urge to expand since, as described above, the distance from longitudinal axis 80 of generally tubular member 80, which generally coincides with the center of tear 280, to second end 200 of the proximal securing member 70 at a rest state of proximal securing member 70 is greater than the radius of tear 280. Thus if tear 280 expands, such as during flexion exercises, proximal securing members 70 expand and remain in contact with annulus 270.

In another embodiment, as illustrated in FIG. 1D, plurality of proximal securing members 70 are situated external of annulus 270. In one embodiment, at least a portion of first face 250 of proximal securing members 70 meet the outer wall of annulus 270. Plurality of proximal securing members 70 are arranged to further secure implant 10 to the outer wall of annulus 270 so as to prevent movement thereof in response to forces applied thereto in directions which differ from the direction of ejection force 290.

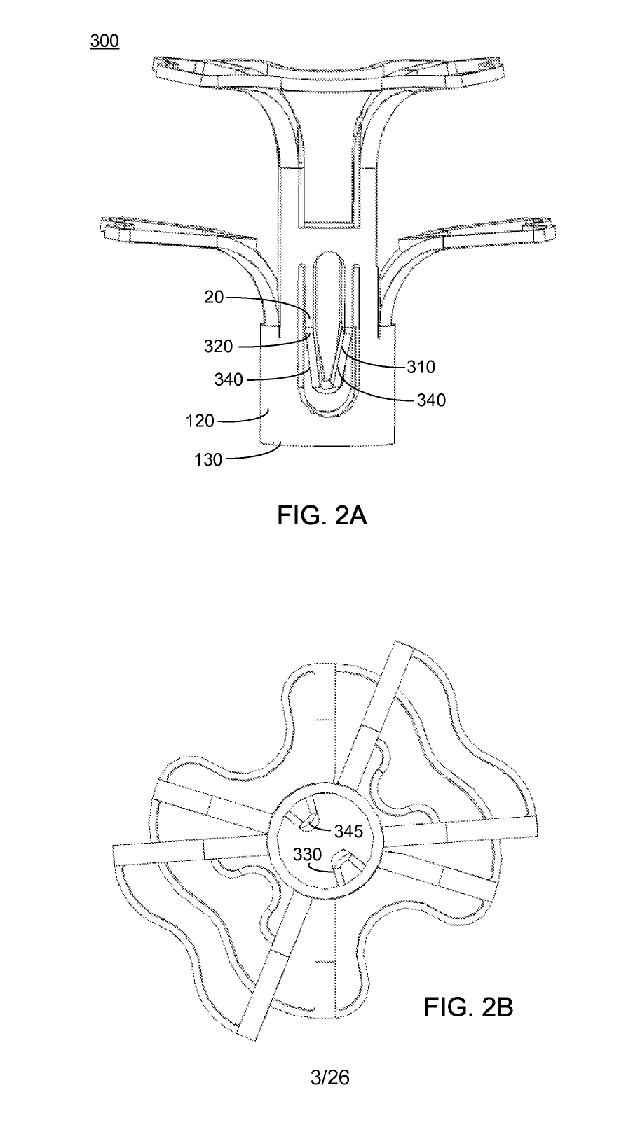

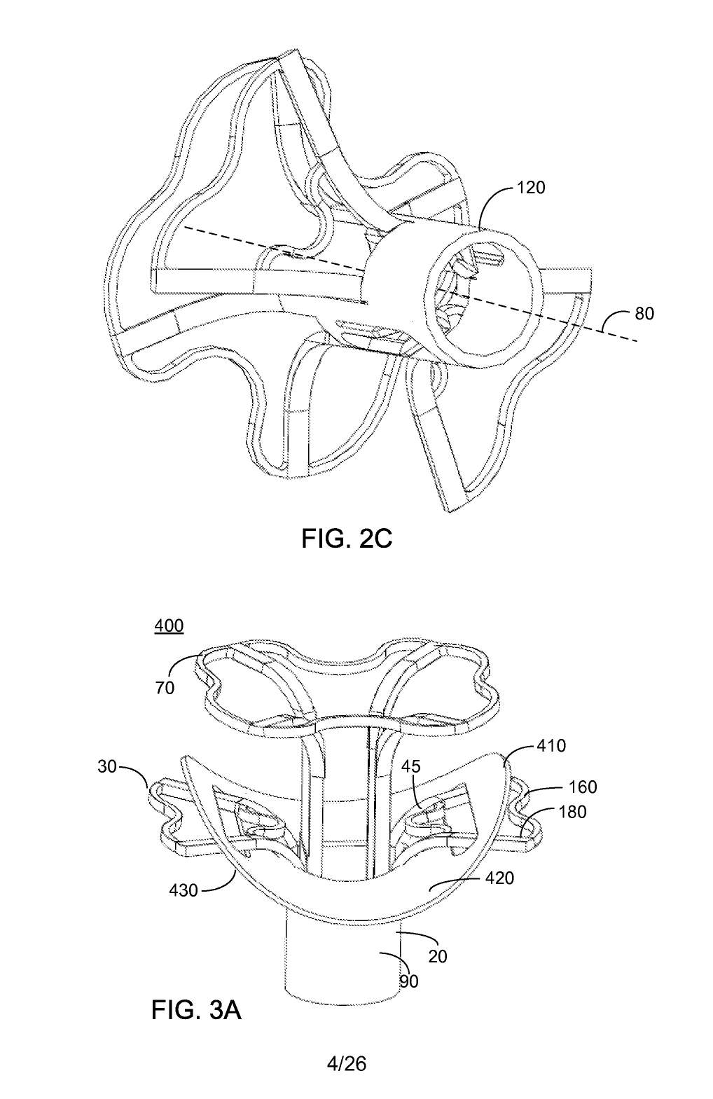

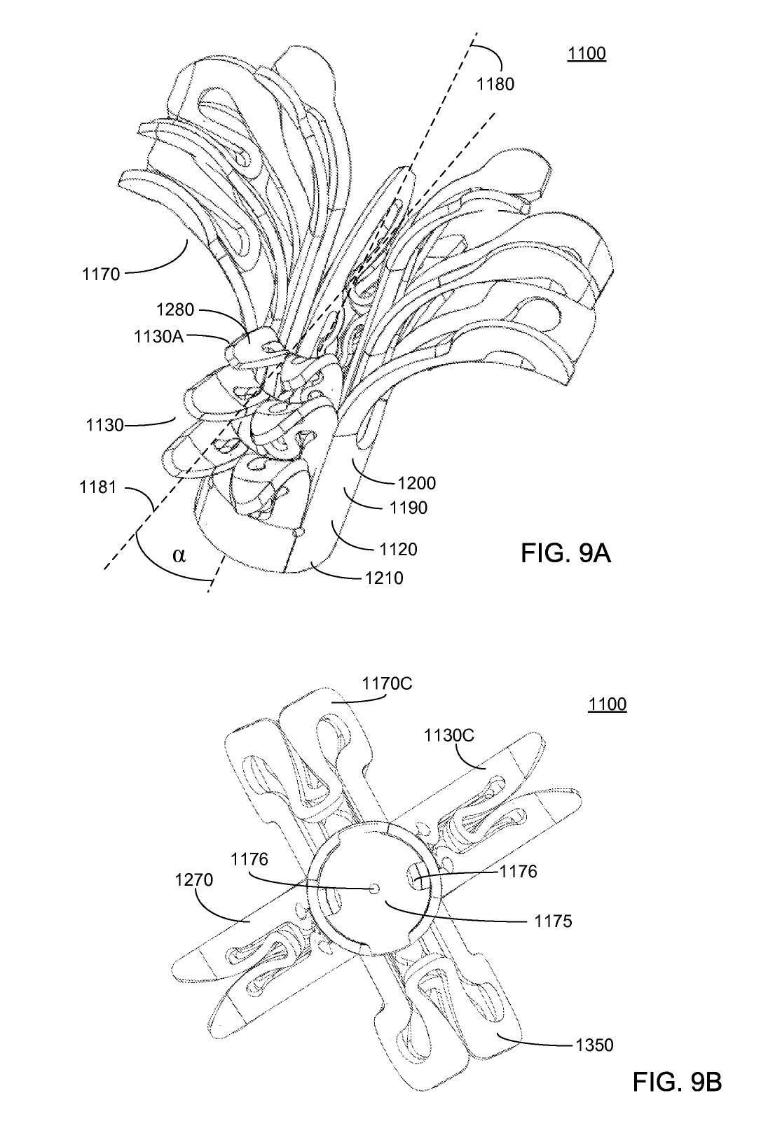

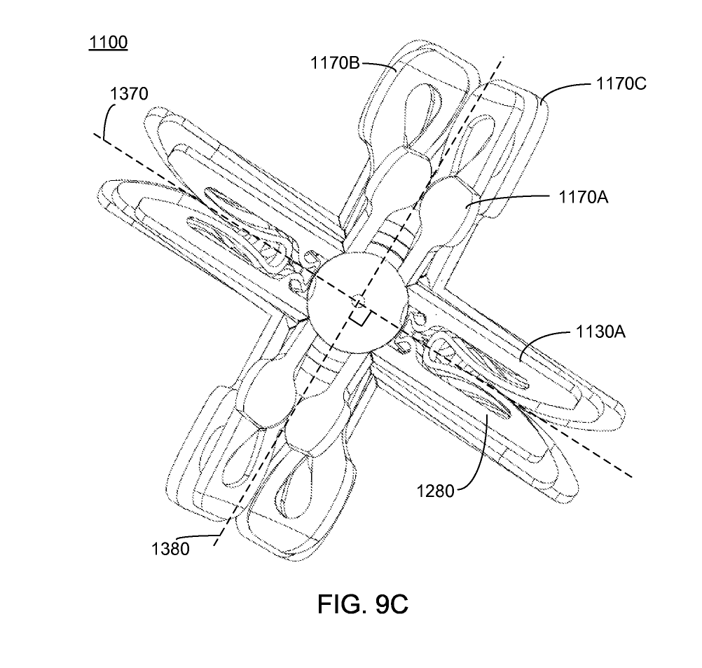

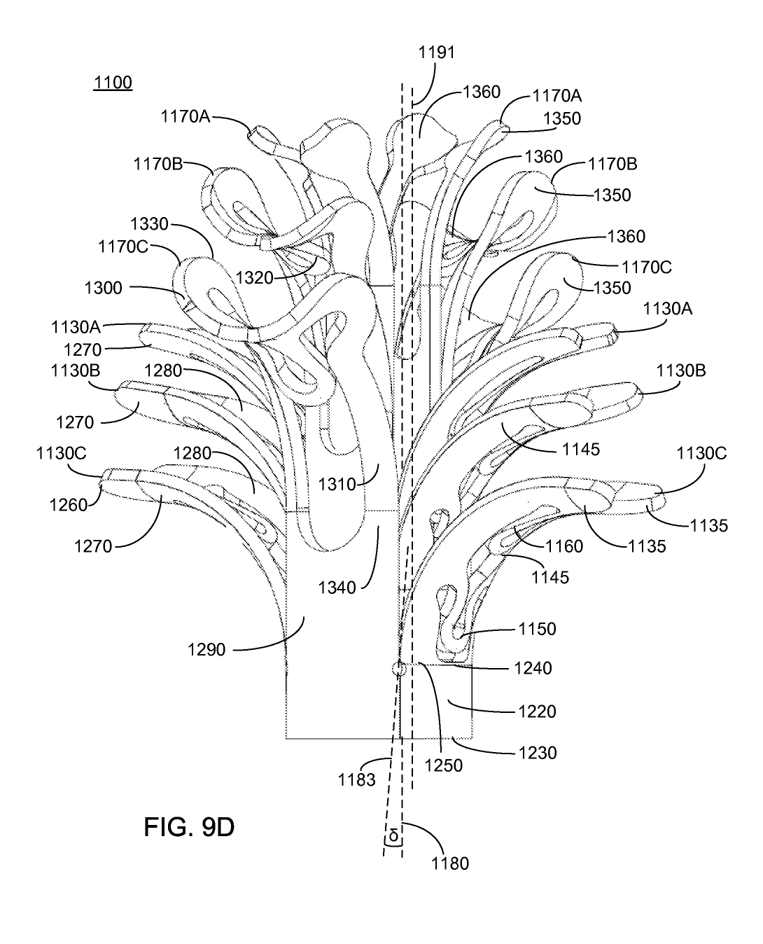

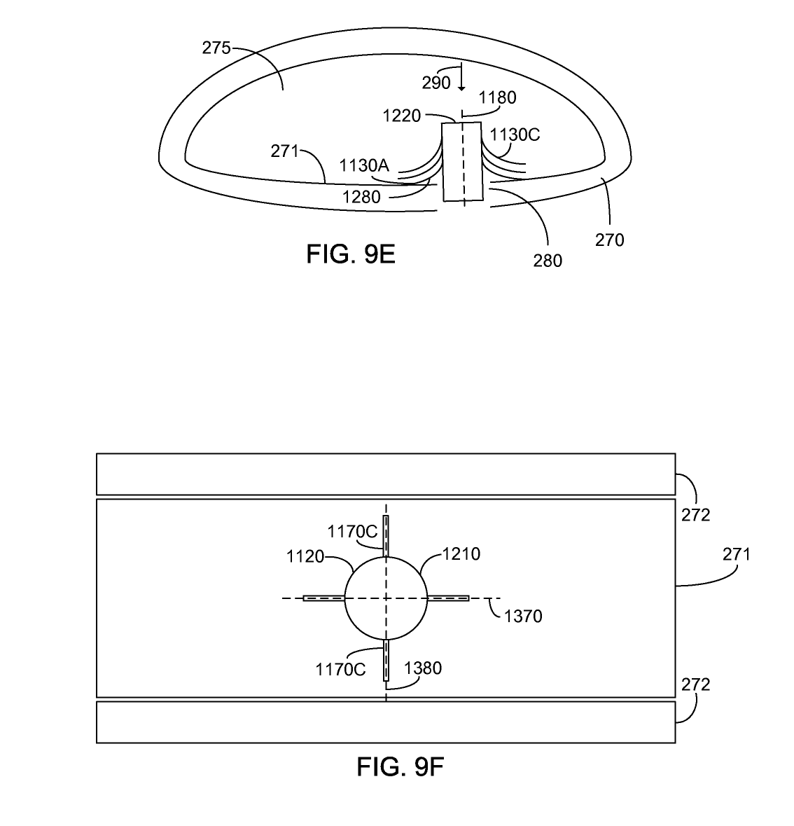

FIG. 2A illustrates a side view of an implant 300 for repair of a spinal inter-vertebral disc, FIG. 2B illustrates a view from a distal end of implant 300 and FIG. 2C illustrates a perspective view of implant 300, implant 300 illustrated in the deployed configuration. Implant 300 is in all respects similar to implant 10 of FIG. 1A, with the exception that implant 300 further comprises a plurality of flow blocking members 310. Each flow blocking members 310 exhibits: a first end 320; and a second end 330, opposing first end 320 and comprising a pair of arms 340 connected by a linking member 345. Plurality of flow blocking members 310 are arranged radially about generally tubular member 20, and may interrupt a portion of axial support member 120 proximal of first end 130 thereof. First end 320 of each flow blocking member 310 is connected to generally tubular member 20 and second end 330 of each flow blocking member 310 extends towards longitudinal axis 80. In one embodiment (not shown), plurality of flow blocking members 310 are connected to axial support member 120. Each linking member 345 connects a pair of arms 340 at a point removed from the connection of the arms 340 to generally tubular member 20. Plurality of flow blocking members 310 are arranged to block the flow of nucleus pulposus through generally tubular member 20. In one embodiment (not shown), a single flow blocking member is provided at first end 130 of generally tubular member 20 as will be described below in relation to FIGS. 9A-9E.

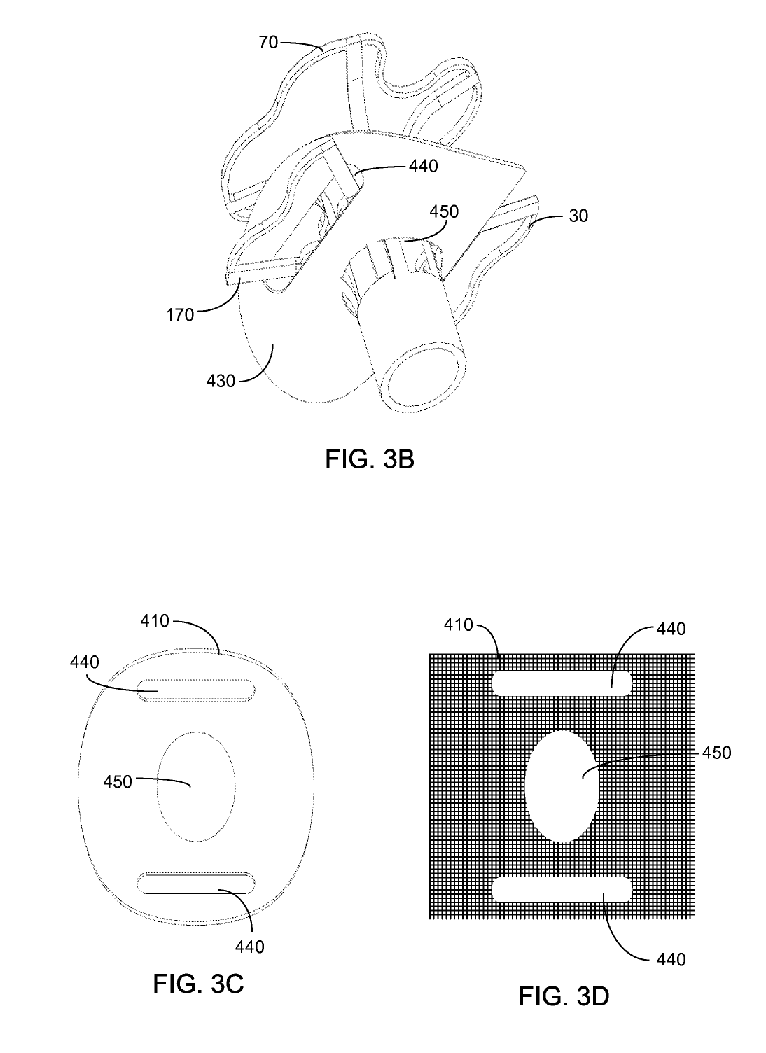

FIG. 3A illustrates a first perspective view of an implant 400 for repair of a spinal inter-vertebral disc in the deployed configuration and FIG. 3B illustrates a second perspective view of implant 400 in the deployed configuration. Implant 400 is in all respects similar to implant 10 of FIG. 1A, with the exception that implant 400 further comprises a barrier material 410. Barrier material 410 exhibits a first face 420 and a second face 430 opposing first face 420. Barrier material 410 comprises: a plurality of slits 440, extending from first face 420 to second face 430; and a generally circular hole 450, extending from first face 420 to second face 430. In one embodiment, as illustrated in FIG. 3C, barrier material 410 is solid and is constituted of a polymer skin. In another embodiment, as illustrated in FIG. 3D, barrier material 410 is constituted of a braided sheet. In another embodiment (not shown), barrier material 410 is constituted of a nano-fiber web. In one embodiment, barrier material 410 is a unidirectional barrier, as described in World Intellectual Property Organization Publication WO 2010/089717, published 12 Aug. 2010. In one particular embodiment, barrier material 410 may be arranged to further act as a scaffold for stem cells or other biological products. In another embodiment, barrier material 410 is replaced with material arranged to serve as a scaffold for stem cells or other biological products. Hole 450 is arranged to mate with generally tubular member 20 and extend over outer surface 90 thereof. In one embodiment, hole 450 exhibits a tight fit over generally tubular member 20 so as to seal outer surface 90. Each of plurality slits 440 is arranged to mate with one inter-annulus support member 30. First face 170 of each inter-annulus support member 30 faces first face 420 of barrier material 410 at link member 45 and second face 180 of each inter-annulus support member 30 faces second face 430 of barrier material 410 at second end 160. As described above in relation to FIGS. 1C-1D, implant 400 is situated in annulus 270 within a tear 280. Barrier material 410 is arranged to prevent the flow of nucleus pulposus through the surgically created channel via any space remaining between implant 400 and the inner walls of tear 280.

The above has been described in relation to barrier material 410 arranged on the distal portion of implant 400, however this is not meant to be limiting in any way. Barrier material may be alternately, or additionally, deployed on the proximal portion of implant 400, without exceeding the scope. In particular, a barrier material may be supplied deployed on proximal securing members 70 of implant 10.

FIG. 4A illustrates a first side view of implant 300 in a delivery configuration and FIG. 4B illustrates a second side view of implant 300 in the delivery configuration.

Plurality of inter-annulus support members 30 and plurality of proximal securing members 70 do not extend past plane 91 defined by outer surface 90 of generally tubular member 20. In one embodiment, implant 300 further comprises a plurality of holes 350 each adjacent to the connection between a particular link member 45 and axial support member 120. Plurality of holes 350 provides for easier transition from the delivery configuration to the deployed configuration described above.

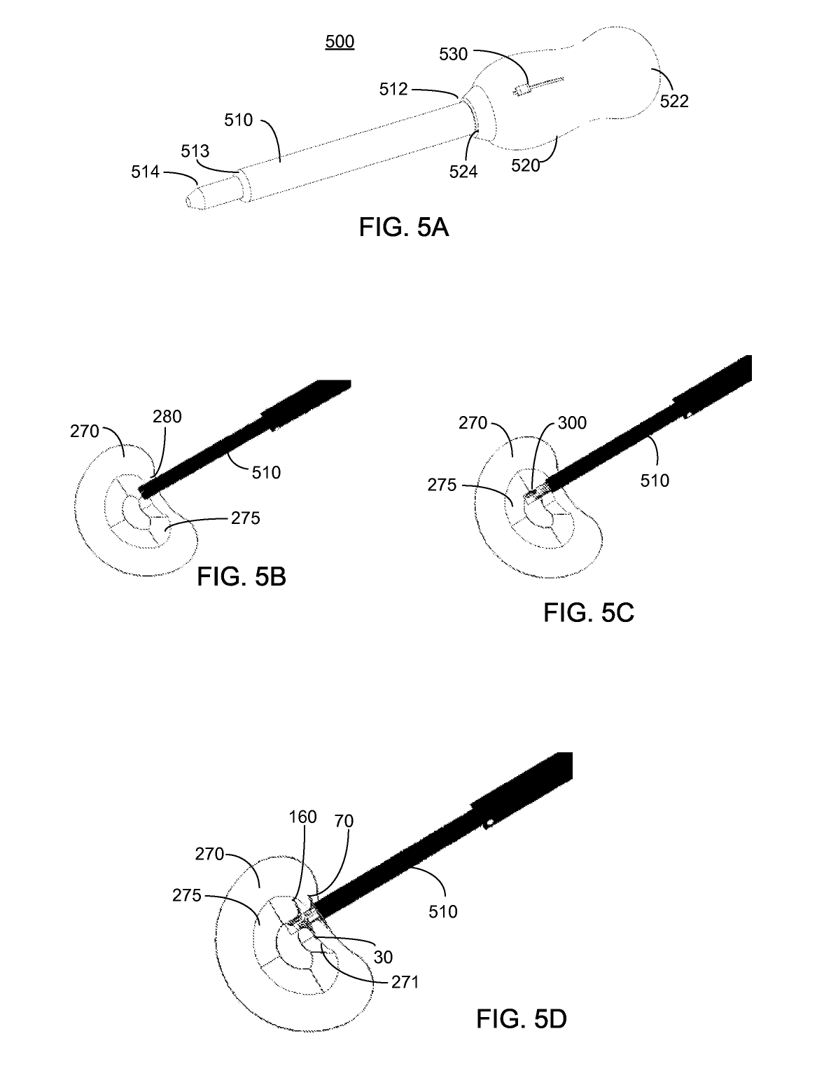

Implant 300 is illustrated in an embodiment wherein flow blocking members 310 are flush with generally tubular member 20, however this is not meant to be limiting in any way. In another embodiment, flow blocking members 310 are in the same position as in the deployed configuration. In one non-limiting embodiment, the distance between second end 160 of each inter-annulus support member 30 and first end 130 of axial support member 120 is 5-25 mm and in one further embodiment the distance is about 9 mm. In one embodiment, plurality of inter-annulus support members 30 and plurality of proximal securing members 70 are held in the delivery configuration by a restraining device, as will be described below in relation to FIGS. 5A-5D. In another embodiment, plurality of inter-annulus support members 30, plurality of proximal securing members 70 and plurality of flow blocking members 310 are each constituted of a shape memory alloy and move from the delivery configuration to the deployed configuration responsive to body heat. The above has been described in relation to implant 300, however this is not meant to be limiting in any way. The delivery configurations of implants 10 and 400 are in all respects similar to the delivery configuration of implant 300.

FIG. 5A illustrates a perspective view of a delivery system 500 for delivering any of implants 10, 300 and 400 into an annulus. For ease of understanding the below will be described in relation to implant 300, however this is not meant to be limiting in any way. FIG. 5B illustrates a perspective view of delivery system 500 with a distal end thereof inserted through tear 280 into area 275 of annulus 270. FIG. 5C illustrates a perspective view of a partial deployment of implant 300 into annulus 270 and FIG. 5D illustrates a perspective view of full deployment of implant 300 into annulus 270. For ease of understanding, FIGS. 5A-5D will be described together. Delivery system 500 comprises: a restraining device, such as a delivery tube 510, exhibiting a proximal end 512 and a distal end 514; a handle 520, exhibiting a proximal end 522 and a distal end 524; and a delivery lever 530. In one embodiment, distal end 514 of delivery tube 510, beginning distally of a ridge 513, is narrower than the rest of delivery tube 510, thereby allowing entry of distal end 514 of delivery tube 510 into tear 280, while allowing for a thicker proximal portion of delivery tube 510 for mechanical stability. Proximal end 512 of delivery tube 510 is connected to distal end 524 of handle 520. In one embodiment, an extension of delivery lever 530 is slideably secured to the side of handle 520 and is arranged to push delivery tube 510 from proximal end 512. In another embodiment (not shown), an extension of delivery lever 530 is attached to proximal end 522 of handle 520.

Implant 300 is situated inside delivery tube 510 in the delivery configuration, as described above in relation to FIGS. 4A-4B. Tear 280 of annulus 270 is surgically expanded to create a channel. Delivery tube 510 is inserted into the surgically created channel of tear 280, as illustrated in FIG. 5B. Advantageously, the diameter of ridge 513 is greater than the diameter of the channel of tear 280 thereby contact of ridge 513 with the outer walls of annulus 270 provides an indication to the user to cease advancement of delivery tube 510, and further prevents deployment of implant 300 at an inappropriate depth within annulus 270. Handle 520 is turned, thus advancing delivery lever 530. As delivery lever 530 advances, it pushes implant 300 out of delivery tube 510, as illustrated in FIG. 5C. As second end 160 of each of plurality of inter-annulus support members 30 exits delivery tube 510, plurality of inter-annulus support members 30 are urged to move from the delivery configuration to the deployed configuration, as described above. In one embodiment, inter-annulus support members 30 are elastically forced into the delivery configuration and inherently urge to return to the deployed configuration. In another embodiment, inter-annulus support members 30 urge to the deployed configuration responsive to body heat. In an embodiment wherein plurality of flow blocking members 310 are each constituted of a shape memory alloy, plurality of flow blocking members 310 move from the delivery configuration to the deployed configuration responsive to body heat, as described above.

Plurality of inter-annulus support members 30 secure implant 300 against inner wall 271 of annulus 270, as described above in relation to FIGS. 1A-1B. Delivery tube 510 is withdrawn from area 275 and withdrawn from over the remainder of implant 300, exposing plurality of proximal securing members 70. Proximal securing members 70 move to the deployed configuration and secure implant 300 to the outer walls of annulus 270, as described above. In one embodiment, proximal securing members 70 are elastically forced into the delivery configuration and inherently urge to return to the deployed configuration. In another embodiment, proximal securing members 70 urge to the deployed configuration responsive to body heat.

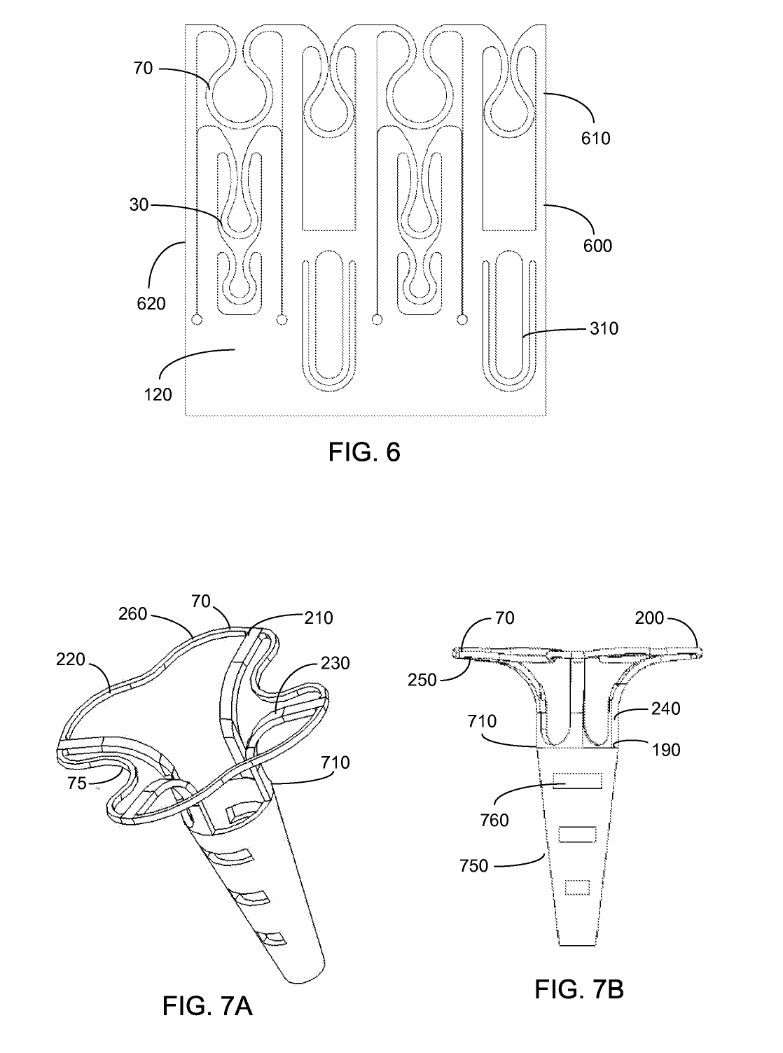

FIG. 6 illustrates one non-limiting embodiment of the production process of implant 300. A sheet 600 of biocompatible material is provided, exhibiting: a first end 610; and a second end 620, opposing first end 610. Sheet 600 is cut such that plurality of inter-annulus support members 30, plurality of proximal securing members 70, axial support member 120 and plurality of flow blocking members 310 remain. Sheet 600 is then rolled up and secured such that first end 610 and second end 620 meet, thereby forming generally tubular member 20. In an exemplary embodiment first end 610 is welded to second end 620 along the entire seam length.

In another embodiment, generally tubular member 20 is provided as a preformed tube of biocompatible material and is cut, preferably by a laser, such that plurality of inter-annulus support members 30, plurality of proximal securing members 70, axial support member 120 and plurality of flow blocking members 310 remain. The above has been described in relation to implant 300, however this is not meant to be limiting in any way and the production of implants 10 and 400 are in all respects similar to the production of implant 300.

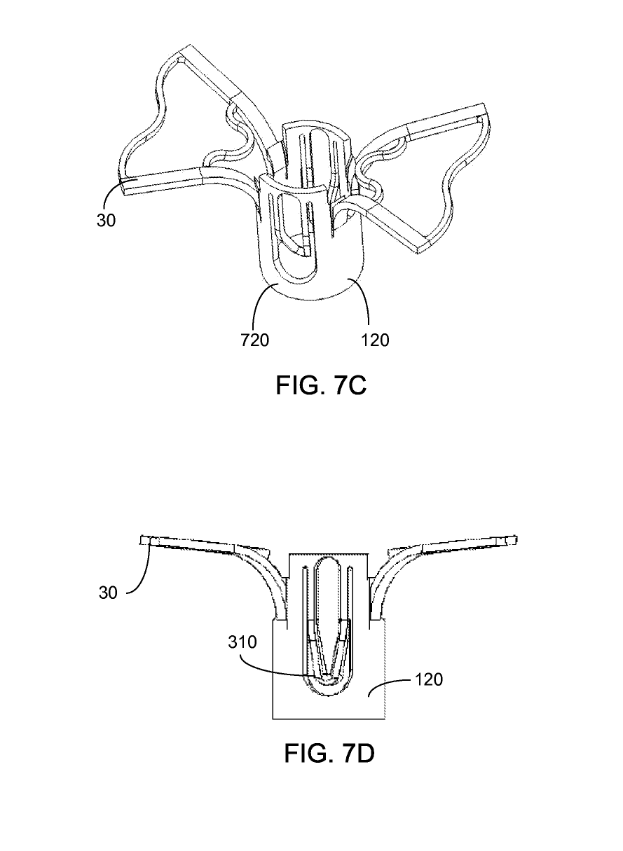

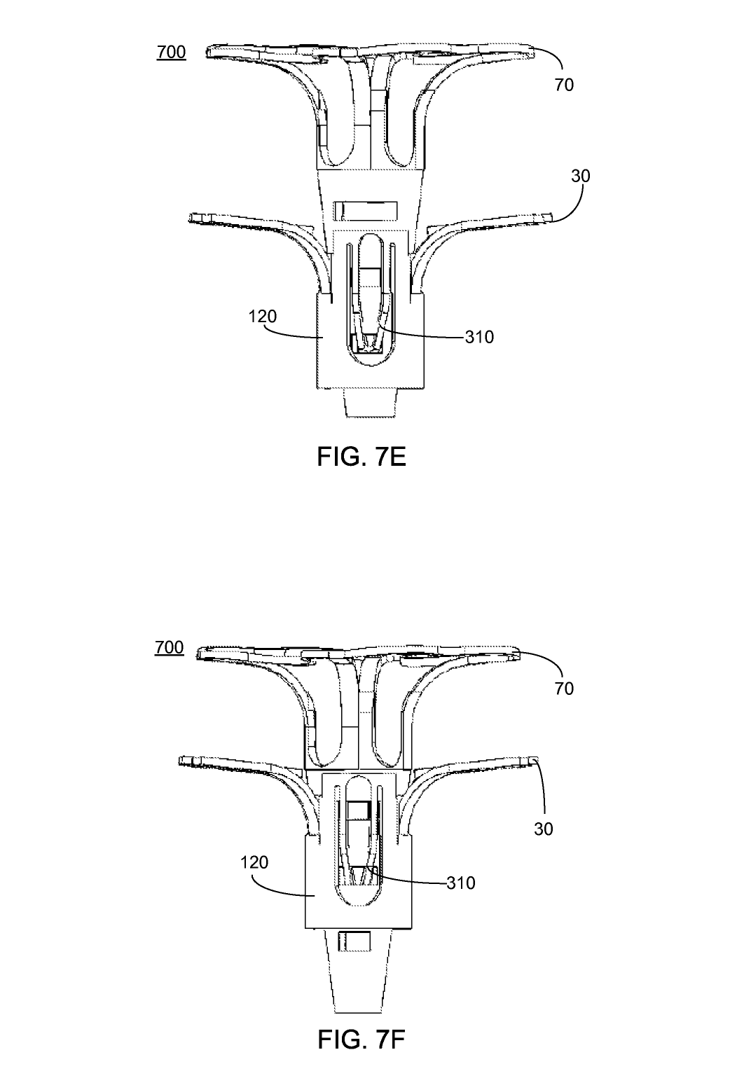

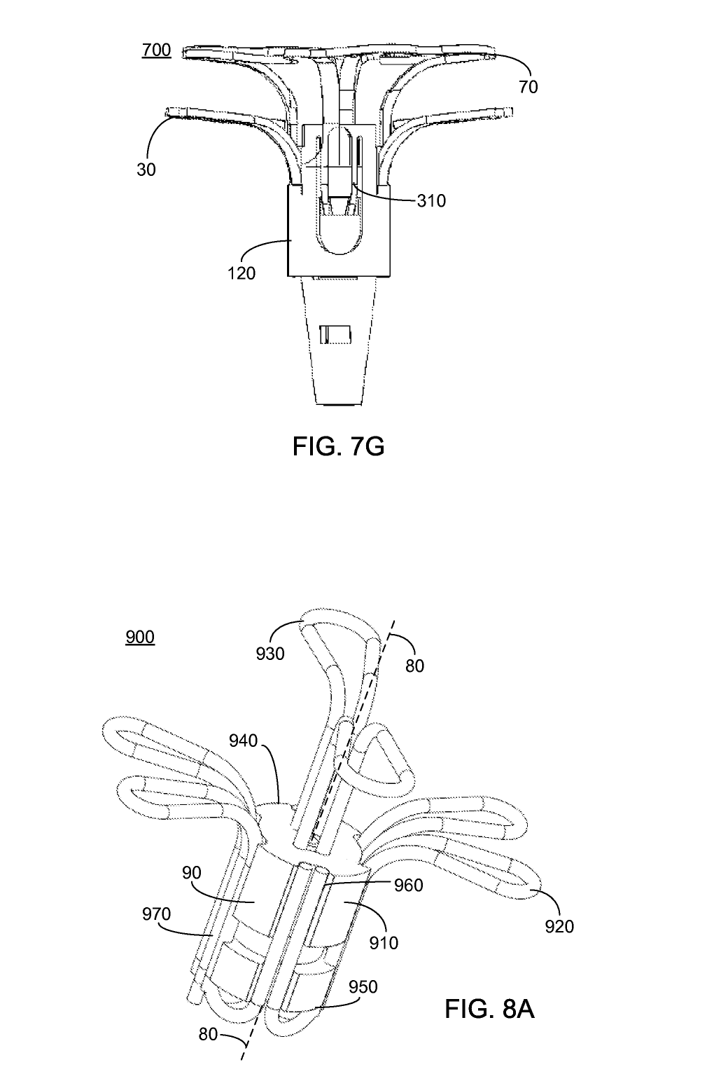

FIGS. 7A-7G illustrate various views and positions of the parts of an implant 700 for repair of a spinal inter-vertebral disc with a variable spacing between inter-annulus support members 30 and proximal securing members 70. Implant 700 is formed of a proximal portion 710 and a distal portion 720. FIG. 7A illustrates a perspective view of proximal portion 710; FIG. 7B illustrates a side view of proximal portion 710; FIG. 7C illustrate a perspective view of distal portion 720; FIG. 7D illustrates a side view of distal portion 720; FIG. 7E illustrates implant 700 with a maximal distance between inter-annulus support members 30 and proximal securing members 70; FIG. 7F illustrates implant 700 with a medium distance between inter-annulus support members 30 and proximal securing members 70; and FIG. 7G illustrates implant 700 with a minimal distance between inter-annulus support members 30 and proximal securing members 70.

Proximal portion 710 comprises a generally conically shaped member 750, exhibiting a plurality of pairs of slits 760 each pair disposed at a respective location along the longitudinal axis of generally conically shaped member 750. Each slit 760 of the pair appears on opposing sides of generally conically shaped member 750, i.e. at a 180° rotation about the longitudinal axis from the other slit 760 of the pair. While pairs of slits 760 are illustrated, this is not meant to be limiting in any way, and three or more slits, preferably at equal rotation angles about the longitudinal axis, at each longitudinal location may be provided without exceeding the scope. While three pairs of slits 760 are illustrated, this is not meant to be limiting in any way, and two slits, or four or more slits may be supplied without exceeding the scope. As described above in relation to FIG. 1A, each proximal securing member 70 comprises an arm 210, an outer link member 220, an inner link member 230 and a proximal extender 240, is connected to adjacent proximal securing members 70 by a proximal connecting member 75 and exhibits a first end 190, a second end 200 opposing first end 190, a first face 250 and a second face 260 opposing first face 250. Proximal extender 240 connects to the proximal end of conically shaped member 750. Distal portion 720 is in all respects similar to implant 300, without proximal securing members 70 attached thereto.

In operation, flow blocking members 310 further provide mechanical securing action to lock distal portion 720 to proximal portion 710 at one of the various slit pairs 760, as illustrated in FIGS. 7E-7G. In particular, flow blocking members 310 are provided and configured to mate with each of the various slit pairs 760, and the desired distance between inner-annulus support members 30 and proximal securing members 70 is selected by mating flow blocking members 310 with the desired slit pair 760. In particular, pressure on proximal portion 710 towards distal portion 720 will variously reduce the distance between inner-annulus support members 30 and proximal securing members 70 in steps, as flow blocking members 310 engage variously with successive slit pairs.

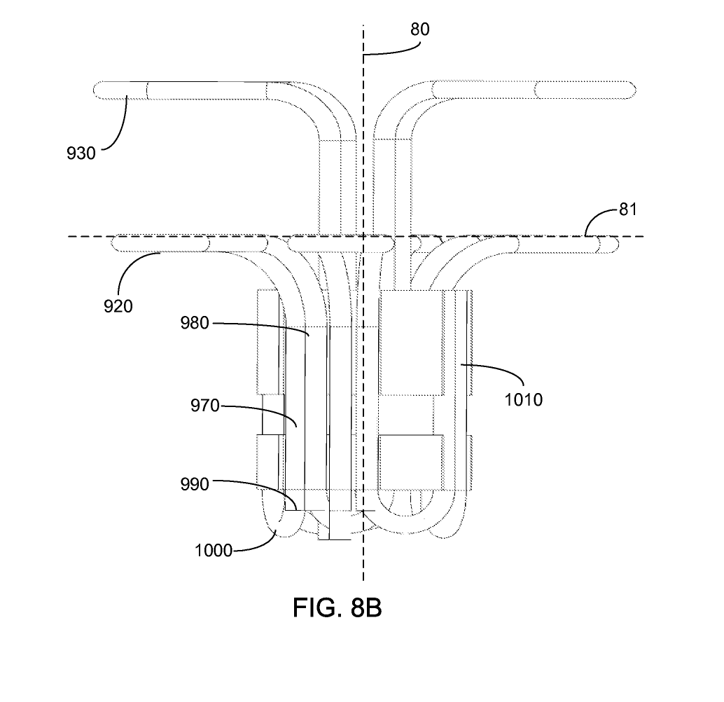

FIG. 8A illustrates a perspective view of an implant 900 for repair of a spinal inter-vertebral disc in a deployed configuration and FIG. 8B illustrates a side view of implant 900 in the deployed configuration, FIGS. 8A and 8B being described together. Implant 900 comprises: a generally tubular member 910; a plurality of inter-annulus support members 920; and a plurality of proximal securing members 930. Generally tubular member 910 exhibits: a longitudinal axis 80; an outer surface 90; a proximal end 940; a distal end 950; a plurality of channels 960 extending from proximal end 940 to distal end 950; a plurality of support wires 970, exhibiting a proximal end 980 and a distal end 990; and a plurality of support wire connectors 1000. Each of plurality of support wires 970 is situated in one of plurality of channels 960, with proximal end 980 facing proximal end 940 of generally tubular member 910 and distal end 990 facing distal end 950 of generally tubular member 910. Each support wire connector 1000 connects distal ends 990 of a pair of support wires 970 situated in adjacent channels 960.

The portion of generally tubular member 910 distal of proximal end 940, plurality of channels 960, plurality of support wires 970 and plurality of support wire connectors 1000 form together a axial support member 1010. In one embodiment, support wires 970 and support wire connectors 1000 are extensions of the wires forming inter-annulus support members 920. In one further embodiment, one or more of support wires 970 and support wire connectors 1000 are extensions of the wires forming proximal securing members 930. In one embodiment, inter-annulus support members 920 and support wire connectors 1000 are formed as a unitary body, thus ensuring maximum resistance to ejection force 290.

Inter-annulus support members 920 are radially arranged about generally tubular member 910, as described above in relation to inter-annulus support members 30. Four inter-annulus support members 920 are illustrated, however this is not meant to be limiting in any way, and more than four inter-annulus support members 920 may be provided, or less than 4 may be provided without exceeding the scope. Inter-annulus support members 920 are formed of wires extending to support wire connectors 1000 and in one embodiment forms a general horseshoe shape in a plane 81, as described above in relation to inter-annulus securing members 30 of FIGS. 1A-1D. In another embodiment (not shown), inter-annulus support members 920 extend distal of plane 81 and exhibit an angle therewith. The dimensions and concavingly curved shape of inter-annulus support members 920 are as described above in relation to inter-annulus securing members 30 and in the sake of brevity will not be further described.

Proximal securing members 930 are radially arranged about generally tubular member 910, as described above in relation to proximal securing members 70. Two proximal securing members 930 are illustrated, however this is not meant to be limiting in any way, and more than two proximal securing members 930 may be provided without exceeding the scope. Proximal securing members 930 are formed of wires extending to support wire connectors 1000 and in one embodiment forms a general horseshoe shape in a plane generally orthogonal to longitudinal axis 80 proximal of plane 81. The dimensions of proximal securing members 930 are as described above in relation to proximal securing members 70 of FIGS. 1A-1D and in the sake of brevity will not be further described.

In operation, and as described above in relation to FIGS. 1C-1D, inter-annulus support members 920 oppose ejection force 290. Additionally and advantageously, ejection force 290 is generally parallel to longitudinal axis 80 and is thus opposed by the linear extension formed by support wire connectors 1000 which would need to experience buckling for inter-annulus support members 920 to significantly bend away from the inner wall, thus providing an increased resistance to ejection force 290 as compared to the purely elastic resistance of prior art devices. Advantageously, the arrangement of implant 900 thus resists ejection force 290 responsive to both buckling and bending resistance, which represents an increased resistance in relation to exclusively bending resistance of the prior art.

FIG. 9A illustrates a perspective view of an implant 1100 for repair of a spinal inter-vertebral disc in a deployed configuration, FIG. 9B illustrates a bottom view of implant 1100, FIG. 9C illustrates a top view of implant 1100, FIG. 9D illustrates a side view of implant 1100, FIG. 9E illustrates a high level schematic diagram of a side cut view of implant 1100 inserted into an annulus 270, and FIG. 9F illustrates a high level schematic diagram of a bottom view of implant 1100 inserted into annulus 270, the figures being described together.

Implant 1100 comprises: a generally tubular member 1120; a plurality of inter-annulus support members 1130; a plurality of proximal securing members 1170; and a flow blocking member 1175. In one embodiment, generally tubular member 1120, plurality of inter-annulus support members 1130, plurality of proximal securing members 1170 and flow blocking member 1175 each comprise a polymeric material. In one embodiment, plurality of inter-annulus support members 1130 and plurality of proximal securing members 1170 each exhibit superelastic properties. In one embodiment, plurality of inter-annulus support members 1130 and plurality of proximal securing members 1170 are each constituted of a shape memory alloy. In one further embodiment, plurality of inter-annulus support members 1130 and plurality of proximal securing members 1170 each comprise Nitinol. In one embodiment, generally tubular member 1120 comprises elastic material. In one embodiment, one or more of generally tubular member 1120, inter-annulus support members 1130 and proximal securing members 1170 comprise a shape memory polymer.

Each inter-annulus support member 1130 comprises a plurality of layers 1130A, 1130B and 1130C. Each of inter-annulus support member layers 1130A, 1130B and 1130C comprises a pair of arms 1135, a pair of link members 1145, an inter-annulus inner connecting member 1150, an inter-annulus outer connecting member 1160 and exhibits: a first end 1250; a second end 1260, opposing first end 1250; a first face 1270; and a second face 1280, opposing first face 1270. Each second face 1280 of inter-annulus support member layers 1130A, 1130B and 1130C is in one embodiment concavingly shaped, as described above in relation to inter-annulus support member 30 of FIGS. 1A-1D, with first faces 1280 of the respective pair of arms 1135 at least partially facing each other. In one embodiment, the thickness of each of inter-annulus support member layers 1130A, 1130B and 1130C, i.e.

the distance between first face 1270 and second face 1280, is 0.05-0.5 mm preferably 0.25 mm. In one embodiment, each layer 1130A, 1130B and 1130C exhibits a different thickness. Three inter-annulus support member layers are illustrated, however this is not meant to be limiting in any way and any number of inter-annulus support member layers may be supplied without exceeding the scope. In one embodiment, 1-12 inter-annulus support member layers are provided for each inter-annulus support member 1130.

Each proximal securing member 1170 comprises a plurality of layers 1170A, 1170B and 1170C. Each of the proximal securing member layers 1170A, 1170B and 1170C comprises: a pair of link members 1310; an optional outer connecting member 1320; a pair of arms 1330; and a pair of proximal extenders 1340, and exhibits: a first end 1290; a second end 1300 opposing first end 1290; a first face 1350; and a second face 1360 opposing first face 1350. In one embodiment, the thickness of each of proximal securing member layers 1170A, 1170B and 1170C, i.e. the distance between first face 1350 and second face 1360, is 0.05-0.5 mm preferably about 0.25 mm. In one embodiment, each layer 1170A, 1170B and 1170C exhibits a different thickness. Three proximal securing member layers are illustrated, however this is not meant to be limiting in any way and any number of proximal securing member layers may be supplied without exceeding the scope. In one embodiment, 1-12 proximal securing member layers are provided for each proximal securing member 1170.

Generally tubular member 1120 exhibits: a longitudinal axis 1180 extending through the center of generally tubular member 1120; an outer surface 1190 defining a plane 1191; a proximal end 1200; a distal end 1210; and a axial support member 1220, formed by a distal portion of generally tubular member 1120 and exhibiting a first end 1230 defining distal end 1210 of generally tubular member 1120 and a second end 1240 facing proximal end 1200 of generally tubular member 1120. In one embodiment, the distance between first end 1230 and second end 1240 of axial support member 1220 is greater than 1 mm. In one non-limiting embodiment, generally tubular member 1120 exhibits a generally circular cross section. In another non-limiting embodiment (not shown), generally tubular member 1120 exhibits a generally elliptic cross section. In another non-limiting embodiment (not shown), generally tubular member 1120 exhibits a generally oval cross section. In another non-limiting embodiment (not shown), generally tubular member 1120 exhibits a generally rectangular cross section.

Flow blocking member 1175 generally covers distal end 1210 of generally tubular member 1120 and in one embodiment comprises one or more holes 1176 arranged to provide porosity for flow blocking member 1175 so as to aid in scar tissue growth. Flow blocking member 1175 is arranged to block the flow of nucleus pulposus through generally tubular member 1120. Additionally, flow blocking member 1175 allows for the placement of regenerative material within axial support member 1220, flow blocking member 1175 preventing the extrusion of the regenerative material from axial support member 1220. In one embodiment, the regenerative material comprises any, or a combination, of stem cells and growth factors, without limitation. In one embodiment (not shown), a plurality of flow blocking members are provided as described above in relation to FIGS. 2A-2C.

First end 1250 of inter-annulus support member layer 1130C is consonant with outer surface 1190, and in particular is in a line with outer surface 1190, is parallel to longitudinal axis 1180 and defines second end 1240 of axial support member 1220. Each of inter-annulus support member layers 1130A and 1130B are coupled to second end 1240 of axial support member 1220. Each of inter-annulus support member layers 1130A, 1130B and 1130C, when deployed, extends radially away from first end 1250 towards second end 1260 generally in a plane 1181. As described above in relation to implant 10 of FIGS. 1A-1D, in operation implant 1100 is delivered through a tear 280 of an annulus 270 and inter-annulus support members 1130 are arranged to meet inner wall 271 of annulus 270. In one embodiment, the angle α between plane 1181 and longitudinal axis 1180, i.e. from first face 1270 of second end 1260 of each inter-annulus support member layer 1130A to longitudinal axis 1180, is 45°-90°. Advantageously, second end 1260 of inter-annulus support member layer 1130A does not meet inner wall 271, thereby when inter-annulus support member layer 1130A presses against inner wall 271 pressure is applied by the curved portion of link members 1145 or the generally flat portion of arms 1135 and not the edge of second end 1260, which could damage inner wall 271. In one embodiment, arms 1135 and link members 1145 are each constructed and arranged such that the contact point of each inter-annulus support member layer 1130A with inner wall 271 is 1-12 mm from the edge of tear 280 and in one further embodiment is about 6 mm from the edge of tear 280.

Inter-annulus support member layers 1130A, 1130B and 1130C are arranged in a layered formation. Specifically, first face 1270 of layer 1130A faces second face 1280 of layer 1130B, first face 1270 of layer 1130B faces second face 1280 of layer 1130C and first face 1270 of layer 1130C generally faces outer surface 1190 of generally tubular member 1120. In one embodiment, the distance between adjacent layers, i.e. layers 1130A and 1130B; and layers 1130B and 1130C, is 0-4 mm, preferably 0.3 mm. In one embodiment, the distance between layers 1130A and 1130B is different than the distance between layers 1130B and 1130C. In one embodiment (not shown), as described above in relation to implant 10 of FIGS. 1A-1E, an angle is defined between link members 1145 of each inter-annulus support member layer 1130A, 1130B and 1130C, optionally the angle being 45°-90°.

Plurality of inter-annulus support members 1130 are arranged radially about generally tubular member 1120. Two inter-annulus support members 1130 are illustrated, however this is not meant to be limiting in any way, and any number of inter-annulus support members 1130 may be provided without exceeding the scope. Each arm 1135 of each of inter-annulus support member layers 1130A, 1130B and 1130C is connected to second end 1240 of axial support member 1220 by a respective link member 1145. Each of plurality of inter-annulus inner connecting members 1150 and plurality of inter-annulus outer connecting members 1160 are arranged to connect a pair of arms 1135 and provide support thereto. Each inter-annulus inner connecting member 1150 and inter-annulus outer connecting member 1160 is in one non-limiting embodiment generally horseshoe shaped and extends generally in the direction of first end 1250. The extension of each inter-annulus outer connecting member 1160 begins at an end of each respective arm 1135 consonant with second end 1260.

As indicated above, second end 1260 of each of inter-annulus support member layers 1130A, 1130B and 1130C extends away from longitudinal axis 1180 of generally tubular member 1120 and extends past plane 1191 defined by outer surface 1190 of generally tubular member 120. In one embodiment, first face 1270 of each inter-annulus support member layer 1130A, 1130B and 1130C concavingly curves away from second end 1240 of axial support member 1220 to plane 1181 defined by arms 1135, and in one embodiment second face 1280 of each inter-annulus support member layer 1130A, 1130B and 1130C convexingly curves away from second end 1240 of axial support member 1220 to plane 1181. In particular, link member 1145 is connected to axial support member 1220 defining second end 1240 thereof, first face 1270 of link member 1145 generally proceeds from second end 1240 of axial support member 1220 along longitudinal axis 1180 into the concave curve and second face 1280 of link member 1145 generally proceeds from second end 1240 of axial support member 1220 along longitudinal axis 1180 into the convex curve. In another embodiment, link member 1145 generally proceeds from second end 1240 of axial support member 1220 along an axis 1183 into the curve. An acute angle δ is defined between axis 1183 and longitudinal axis 1180, acute angle δ being defined from second face 1280 of link member 1145 to longitudinal axis 1180. Thus, inter-annulus support member layers 1130A, 1130B and 1130C are each at all times preferably proximal of a straight line drawn from first end 1250 to second end 1260 of the respective inter-annulus support member layer 1130A, 1130B, 1130C.

In one embodiment, link members 1145 and axial support member 1220 are formed as a unitary body, thus ensuring maximum resistance to ejection forces applied thereto. In an exemplary embodiment, plurality of inter-annulus support members 1130 are formed as part of generally tubular member 1120. In one embodiment, plurality of proximal securing members 1170 are formed as part of generally tubular member 1120. In one embodiment, plurality of proximal securing member layers 1170C are displaced from plurality of inter-annulus support member layers 1130A along longitudinal axis 1180 by the thickness of annulus 270, as described above in relation to FIG. 1D. In another embodiment, plurality of proximal securing member layers 1170C are displaced from plurality of inter-annulus support member layers 1130A along longitudinal axis 1180 by less than the thickness of annulus 270, as described above in relation in FIG. 1C.

First end 1290 of each proximal securing member layer 1170C is consonant with outer surface 1190, and in particular is in a line with outer surface 1190, and is parallel to longitudinal axis 1180. Each of proximal securing member layers 1170A, 1170B and 1170C extends radially away from first end 1290 towards second end 1300. Each proximal extender 1340 extends proximally from second end 1240 of axial support member 1220 in a line generally parallel with longitudinal axis 1180, in the plane defined by outer surface 1190, to a first end of the respective link member 1310. First face 1350 of each link member 1310 exhibits a curve extending in a direction generally away from the respective proximal extender 1340 and extending into a first end of the respective arm 1330, generally faces axial support member 1220 when in the deployed configuration and is concavingly curved from second end 1240 of axial support member 1220. Particularly, in one embodiment, first face 1350 of each link member 1310 generally proceeds from the respective proximal extender 1340 along longitudinal axis 1180 into the concave curve. In one embodiment, each arm 1330 of each proximal securing member layers 1170A, 1170B and 1170C extends generally into a plane orthogonal to longitudinal axis 1180 and proximal of plane 1181. A second end of each arm 1330, opposing the first end thereof, of the respective one of proximal securing member layers 1170A, 1170B and 1170C is connected to a respective optional outer connecting member 1320. In one embodiment, link members 1310 comprise an elastic material and link members 1310 and arms 1330 are each constructed and arranged such that in an at rest state of the respective proximal securing member layer 1170A, 1170B, 1170C the distance between second end 1300 of each proximal securing member layer 1170A, 1170B and 1170C and longitudinal axis 1180 is 0.5-3 mm greater than the radius of tear 280 of annulus 270, preferably about 1.5 mm greater than the radius of tear 280.

Each of proximal securing member layers 1170A, 1170B and 1170C is in one embodiment concavingly shaped, with second faces 1360 of the respective pair of arms 1310 at least partially facing each other. Proximal securing member layers 1170A, 1170B and 1170C of each proximal securing member 1170 are arranged in a layered formation. Specifically, first face 1350 of layer 1170A faces second face 1360 of layer 1170B, first face 1350 of layer 1170B faces second face 1360 of layer 1170C and first face 1350 of layer 1170C generally faces outer surface 1190 of generally tubular member 1120. In one embodiment, the distance between adjacent layers, i.e. layers 1170A and 1170B; and layers 1170B and 1170C, is 0-4 mm, preferably about 0.3 mm. In one embodiment, the distance between layers 1170A and 1170B is different than the distance between layers 1170B and 1170C.

Plurality of proximal securing members 1170 are arranged radially about generally tubular member 1120 proximal of plurality of inter-annulus support members 1130 and first end 1290 of each proximal securing member 1170 is connected to a proximal extension of generally tubular member 1120. Two proximal securing members 1170 are illustrated, however this is not meant to be limiting in any way, and fewer, or more, proximal securing members may be provided without exceeding the scope.

As described above, inter-annulus support member layers 1130A extend into a plane 1181 and proximal securing member layers 1170A extend into a plane proximal of inter-annulus support member layers 1130A, in one embodiment generally orthogonal to longitudinal axis 1180. In one embodiment, inter-annulus support member layers 1130A extend generally along a support member axis 1370 situated within plane 1181 and proximal securing member layers 1170A extend generally along a securing member axis 1380, which is rotated from support member axis 1370 about longitudinal axis 1180, preferably by 90°. As described above, inter-annulus support member layers 1130B and 1130C are arranged in a layered formation with inter-annulus support member layer 1130A and proximal securing members 1170B and 1170C are arranged in a layered formation with proximal securing member layers 1170A.

As illustrated in FIGS. 9E-9F, annulus 270 exhibits a tear 280, optionally comprising a surgically created channel created as part of the discectomy, and implant 1100 is inserted through tear 280 into area 275 of annulus 270. As illustrated in FIG. 9E, axial support member 1220 and plurality of inter-annulus support members 1130 are situated within the enclosed area of annulus 270, i.e. in an area 275 comprising the nucleus pulposus.

Second face 1280 of each inter-annulus support member layer 1130A meets inner wall 271 of annulus 270. In one embodiment (not shown), the portion of generally tubular member 1120 extending along longitudinal axis 1180 proximal of plurality of inter-annulus support members 1130 and plurality of proximal securing members 1170 are situated within tear 280. In one embodiment (not shown), one or more of each proximal securing member layer 1170A, 1170B and 1170C are situated external of annulus 270, preferably in contact with the outer wall of annulus 270. Advantageously, the construction of inter-annulus support members 1130 and proximal securing members 1170 is porous thereby scar tissue grows into and around inter-annulus support members 1130 and proximal securing members 1170 thus affixing implant 1100 to annulus 270 over time.

When ejection force 290 is applied from area 275 to implant 1100, implant 1100 is urged to move proximally through tear 280. Plurality of inter-annulus support member layers 1130A pressed against inner wall 271 of annulus 270 oppose the ejection forces. Additionally and advantageously, ejection force 290 is generally parallel to longitudinal axis 1180 and thus the shape of first face 1270 of link members 1145, which as described above is in one embodiment concave, and the connection to axial support member 1220 significantly increases the resistance to ejection force 290 as compared to the purely elastic resistance of prior art systems, since any significant bending of link members 1145 past the plane orthogonal to longitudinal axis 1180 responsive to ejection force 290 would result in buckling at the connection point between axial support member 1220 and link members 1145. Advantageously, the arrangement of implant 1100 thus resists ejection force 290 responsive to both buckling and bending resistance, which represents an increased resistance in relation to exclusively bending resistance of the prior art.

Additionally, in the embodiment where there is a distance between adjacent layers of each inter-annulus support member 1130, if ejection force 290 is strong enough layer 1130A bends until reaching layer 1130B. Layer 1130B is then bent until reaching layer 1130C which then further bends to resist ejection force 290. In such an embodiment, and in the embodiment where adjacent layers of each inter-annulus support member 1130 are in contact with each other, the layer arrangement of inter-annulus support member 1130 forms a leaf spring type arrangement and thus provides for improved resistance to ejection force 290 while allowing for a more compliant single layer. The stiffness of a leaf spring is given as:

k=(E*n*b*t³)/(6*L³) EQ. 1

where ‘E’ is Young's modulus of the leaves, ‘n’ is the number of leaves, ‘b’ is the width of the leaves, ‘t’ is the thickness of the leaves and ‘L’ is the length of the leaves. As can be seen by EQ. 1, the more leaves in the spring the greater the stiffness. Thus, the use of multiple layers achieves an increased resistance to ejection responsive to ejection force 290, while allowing for the use of a thinner, more compliant material per layer.

In one embodiment, as described above, second face 1280 of each inter-annulus support member layer 1130A comes in contact with inner wall 271 of annulus 270 at a distance of 1-12 mm from the edge of tear 280. Advantageously, as described above in relation to FIGS. 1C-1D, this provides for contact of inter-annulus support members 1130 with healthier tissue thus avoiding damage to the wall of annulus 270 in the vicinity of tear 280. Additionally, due to the shape of inter-annulus support member layers 1130A, which as described above is in one embodiment concave, responsive to ejection force 290 applied to implant 1100, inter-annulus support member layers 1130A extend further along inner wall 271 of annulus 270 thereby further distancing themselves from tear 280 and applying pressure to a more healthier portion of annulus 270. Plurality of inter-annulus inner connecting members 1150 and plurality of inter-annulus outer connecting members 1160 are arranged to prevent unnecessary movement of the respective arms 1135 within annulus 270.

Friction between layers of proximal securing members 1170 and the inner wall of tear 280 prevents movement of implant 1100, particularly responsive to forces applied thereto in directions which differ from the direction of ejection force 290. In one embodiment, as described above, the distance between second end 1300 of each proximal securing member layer 1170A, 1170B and 1170C and longitudinal axis 1180 of generally tubular member 1120, which generally coincides with the center of tear 280, in the at rest state is greater than the radius of tear 280. Therefore, in the deployed configuration, proximal securing member layers 1170A, 1170B and 1170C are not at rest, but instead urge to expand. Thus, if tear 280 expands, such as during flexion exercises, proximal securing member layers 1170A, 1170B and 1170C expand and remain in contact with annulus 270. In an embodiment where one or more layers of proximal securing members 1170 are situated external of annulus 270, and in the embodiment where plurality of proximal securing member layers 1170C are displaced from plurality of inter-annulus support member layers 1130A along longitudinal axis 1180 by at least the thickness of annulus 270 such that proximal securing members 1170 are completely situated external of annulus 270, first face 1350 of the layer adjacent to the outer wall of annulus 250 further secures implant 1100 to the outer wall of annulus 270 so as to prevent movement thereof in response to forces applied thereto in directions which differ from the direction of ejection force 290. As described above in relation to inter-annulus support members 1130, the layer configuration of proximal securing members 1170 provides for increasing resistance to various forces while allowing for the use of a thinner, more compliant material per layer.

As described above, in one embodiment inter-annulus support member layers 1130A extend generally along support member axis 1370 and proximal securing member layers 1170A extend generally along securing member axis 1380 which is rotated from support member axis 1370 about longitudinal axis 1180, preferably by 90°. Preferably, implant 1100 is positioned such that securing member axis 1380 is generally orthogonal to the vertebrae 272 adjacent annulus 270, as illustrated in FIG. 9F. Advantageously, proximal securing members 1170 which are situated within tear 280 deliver force against the tissue within tear 280 in the directions of vertebrae 1390 and not in other directions which could result in expansion of tear 280. Additionally, in the embodiment where link members 1145 exhibit an angle of 45°-90° between each other (not shown) as described above in relation to implant 10, the points of contact of each inter-annulus support member layer 1130A with inner wall 271 are displaced from support member axis 1370 in the superior and inferior directions, thereby presenting inter-annulus support members 1130 with a thicker portion of annulus 270.

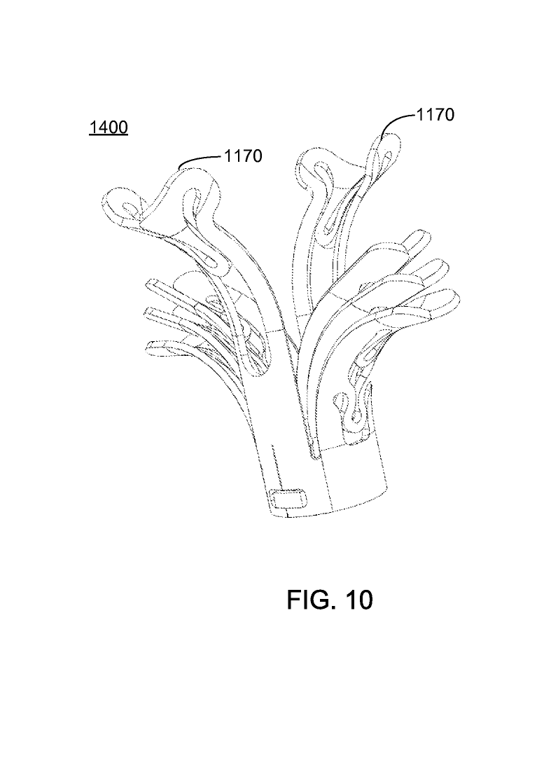

FIG. 10 illustrates a perspective view of an implant 1400 for repair of a spinal inter-vertebral disc in a deployed configuration. Implant 1400 is in all respects similar to implant 1100 of FIGS. 9A-9E with the exception that proximal securing members 1170 each comprise a single layer.

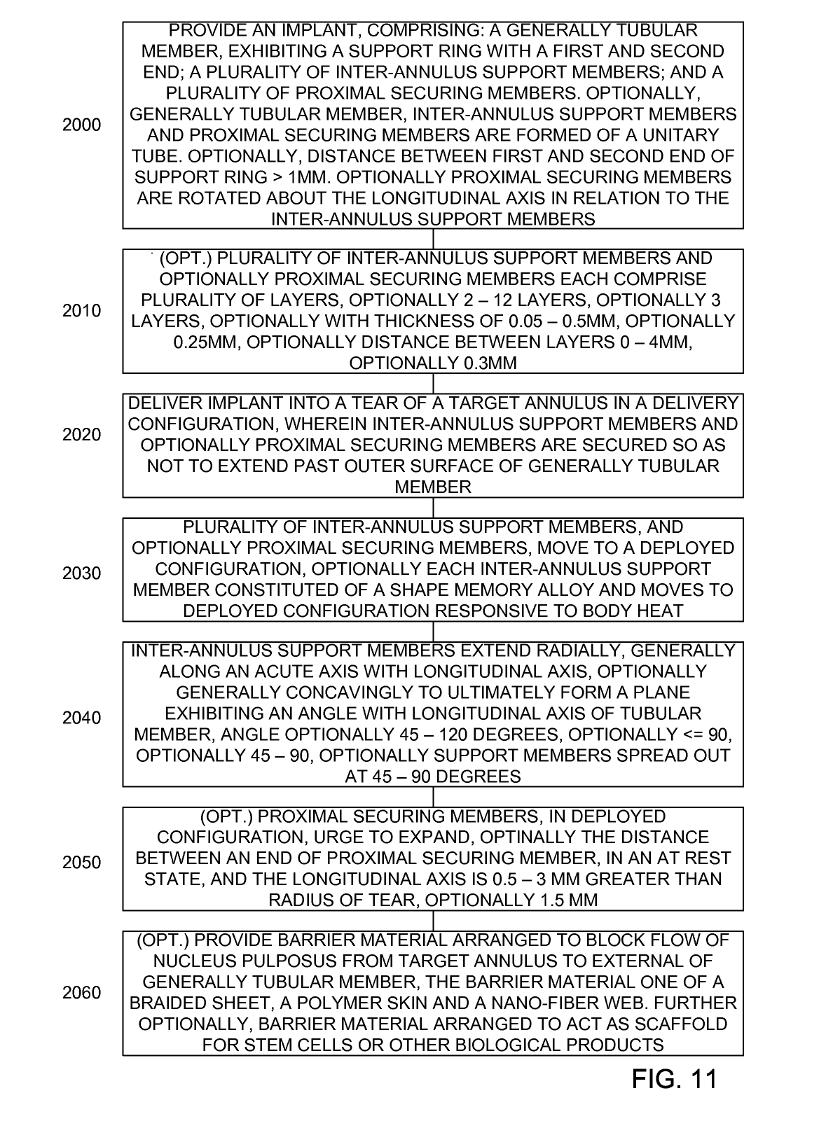

FIG. 11 illustrates a high level flow chart of a first method for repairing a spinal inter-vertebral disc. In stage 2000, an implant is provided, such as one of implants 10, 300 and 400. The implant comprises: a generally tubular member, exhibiting a axial support member with a first end and a second end opposing the first end; a plurality of inter-annulus support members, such as inter-annulus support members 30, 920 or 1120; and a plurality of proximal securing members, such as proximal securing members 70, 930 or 1170. Optionally, the generally tubular member, the plurality of inter-annulus support members and the plurality of proximal securing members are formed of a unitary tube, thus ensuring maximum resistance to ejection forces, as described above. Optionally, the distance between the first end and the second end of the axial support member is greater than 1 mm. In one embodiment, the implant further comprises at least one flow blocking member, such as flow blocking members 310 of implant 300 or flow blocking member 1175 of implant 1100. Optionally, the proximal securing members are rotated about the longitudinal axis of the implant in relation to the inter-annulus support member of the implant.

In optional stage 2010, the plurality of inter-annulus support members of stage 2000 and optionally the plurality of proximal securing members of the implant of stage 2000 each comprise a plurality of layers, as described above in relation to implant 1100. Optionally, each inter-annulus support member and each proximal securing member comprises 2-12 layers, preferably 3 layers. Optionally, the thickness of each layer of each inter-annulus support member and each proximal securing member is 0.05-0.5 mm, preferably 0.25 mm. Optionally, the thickness of each layer can differ from other layers. Optionally, the distance between each adjacent layer is 0-4 mm, preferably 0.3 mm. Optionally, the distance between each pair of adjacent layers can differ from other pairs of adjacent layers.

In stage 2020, the implant of stage 2000 and/or optional stage 2010 is delivered into a tear in a target annulus in a delivery position, wherein the plurality of inter-annulus support members, and optionally the plurality of proximal securing members, are secured so as to not extend past the plane defined by the outer surface of the generally tubular member. In stage 2030, the plurality of inter-annulus support members of stage 2000, and optionally the plurality of proximal securing members of stage 2000, move to a deployed configuration. Optionally, each of the plurality of inter-annulus support members is constituted of a shape memory alloy and moves to the deployed configuration responsive to body heat. Further optionally, each of the plurality of proximal securing members is constituted of a shape memory alloy and moves to the deployed configuration responsive to body heat.

In stage 2040, each of the plurality of inter-annulus support members of stage 2000, in the deployed configuration of stage 2030, extends radially outward, in one embodiment into a generally concavingly shaped form whose end portion defines a plane exhibiting an angle with a longitudinal axis extending through the center of the generally tubular member of stage 2000. Specifically, the angle is defined from the face of each inter-annulus support member facing the axial support member of the generally tubular member.

In one embodiment, the angle is 45°-120°. In one embodiment, the angle is less than, or equal to 90°, optionally 45°-90°. In one embodiment, each inter-annulus support member extends along an axis into the generally concavingly shaped form, the axis exhibiting an acute angle with the longitudinal axis of the generally tubular member, the angle being defined from the longitudinal axis of the generally tubular member to the face of the inter-annulus support member facing away from the axial support member. In one embodiment, each of the plurality of inter-annulus support members of stage 2000 comprises a pair of link members, each coupled to the second end of the axial support member. Optionally, the pair of link members exhibit between each other an angle of 45°-90°, further optionally an angle of about 80°.

In optional stage 2050, each of the plurality of proximal securing members of stage 2000, in the deployed configuration of stage 2030, urge to expand. Optionally, each proximal securing member extends from a first end to a second end thereof, the distance between the second end thereof and the longitudinal axis of stage 2040, in an at rest state of the proximal securing member is 0.5-3 mm greater than the radius of the annulus tear of stage 2020, optionally 1.5 mm greater than the radius of the annulus tear.

In optional stage 2060, a barrier material is further provided and is arranged to block flow of nucleus pulposus from within the target annulus to external of the generally tubular member. In one embodiment, the barrier material is constituted of one of: a braided sheet, a polymer skin, and a nano-fiber web. Further optionally, the barrier material is arranged to act as a scaffold for stem cells or other biological products. As described above, in another embodiment, the barrier material is replaced with material arranged to serve as a scaffold for stem cells or other biological products.

In one embodiment, the various embodiments of implants described above are implemented of a biodegradable material, thus exiting the patient body after a predetermined period.

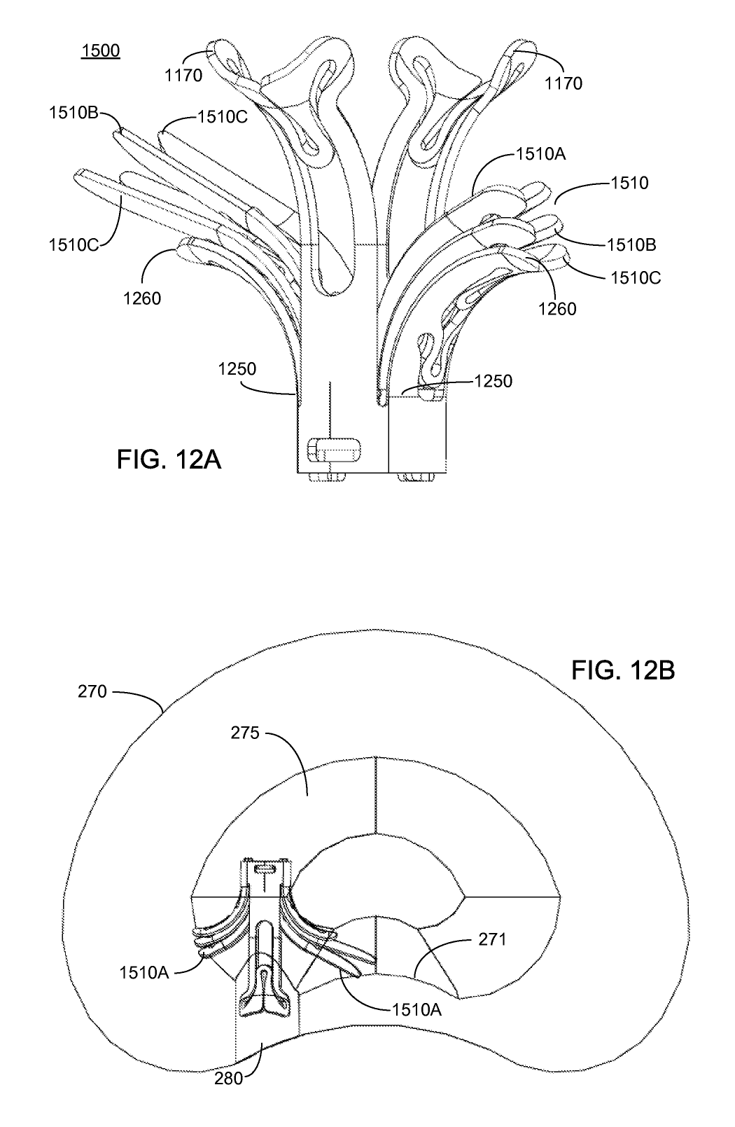

FIG. 12A illustrates a side view of an implant 1500 for repair of a spinal inter-vertebral disc in a deployed configuration; and FIG. 12B illustrates a high level schematic view of implant 1500 inserted in a spine, FIGS. 12A and 12B being described together. Implant 1500 is in all respects similar to implant 1100 of FIGS. 9A-9E with the exception that the plurality of inter-annulus support members 1130 are replaced with a plurality of inter-annulus support members 1510. Proximal securing members 1170 are illustrated as comprising only a single layer, however this is not meant to be limiting in any way and any number of layers may be provided as described above in relation to FIGS. 9A-9E.

Inter-annulus support members 1510 are in all respects similar to inter-annulus support members 1130, with the exception that the layers of each inter-annulus support members 1510 are of different lengths. In particular, in one illustrated embodiment, each inter-annulus support member 1510 comprises a plurality of layers 1510A, 1510B and 1510C, each extending from a first end 1250 to a second end 1260. The distance between first end 1250 and second end 1260 of layers 1510A and 1510B of a first inter-annulus support member 1510 is greater than the distance between first end 1250 and second end 1260 of layers 1510A and 1510B of a second inter-annulus support member 1510. The distance between first end 1250 and second end 1260 of layer 1510C of the first inter-annulus support member 1510 is illustrated as being substantially equal to the distance between first end 1250 and second end 1260 of layer 1510C of the second inter-annulus support member 1510, however this is not meant to be limiting in any way and in another embodiment the distance between first end 1250 and second end 1260 of layer 1510C of the first inter-annulus support member 1510 is greater than the distance between first end 1250 and second end 1260 of layer 1510C of the second inter-annulus support member 1510. In another embodiment, only layer 1510A of first inter-annulus support member 1510 is longer than the respective layer of second inter-annulus support member 1510.

Two inter-annulus support members 1510 are illustrated, each comprising three layers, however this is not meant to be limiting in any way and any number of inter-annulus support members 1510 can be provided, each comprising any number of layers, with one or more layers of at least one inter-annulus support member 1510 exhibiting a length different than the length of a corresponding layer of another inter-annulus support member 1510.

In operation, as described above in relation to FIGS. 9A-9E implant 1500 is inserted through tear 280 into area 275 of annulus 270. As a result of the different shape of inner wall 271 of annulus 270 on either side of tear 280, the unsymmetrical lengths of inter-annulus support members 1510 allow each layer 1510A to be in contact with inner wall 271 of annulus 270 with greater uniformity than occurs in the event of symmetrical lengths of inter-annulus support members 1510.

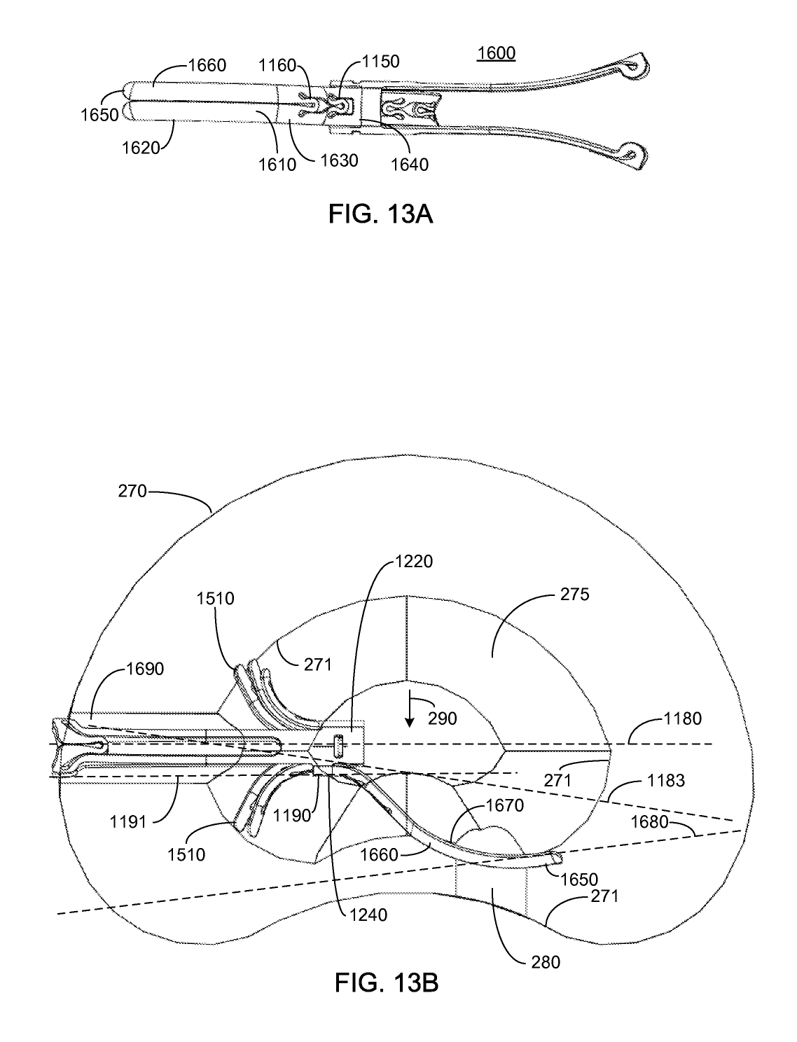

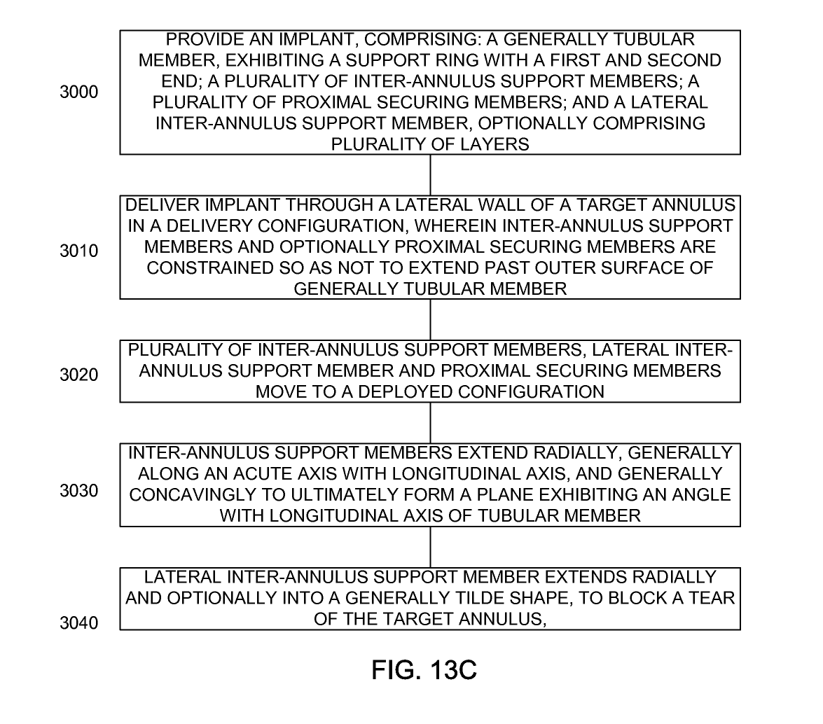

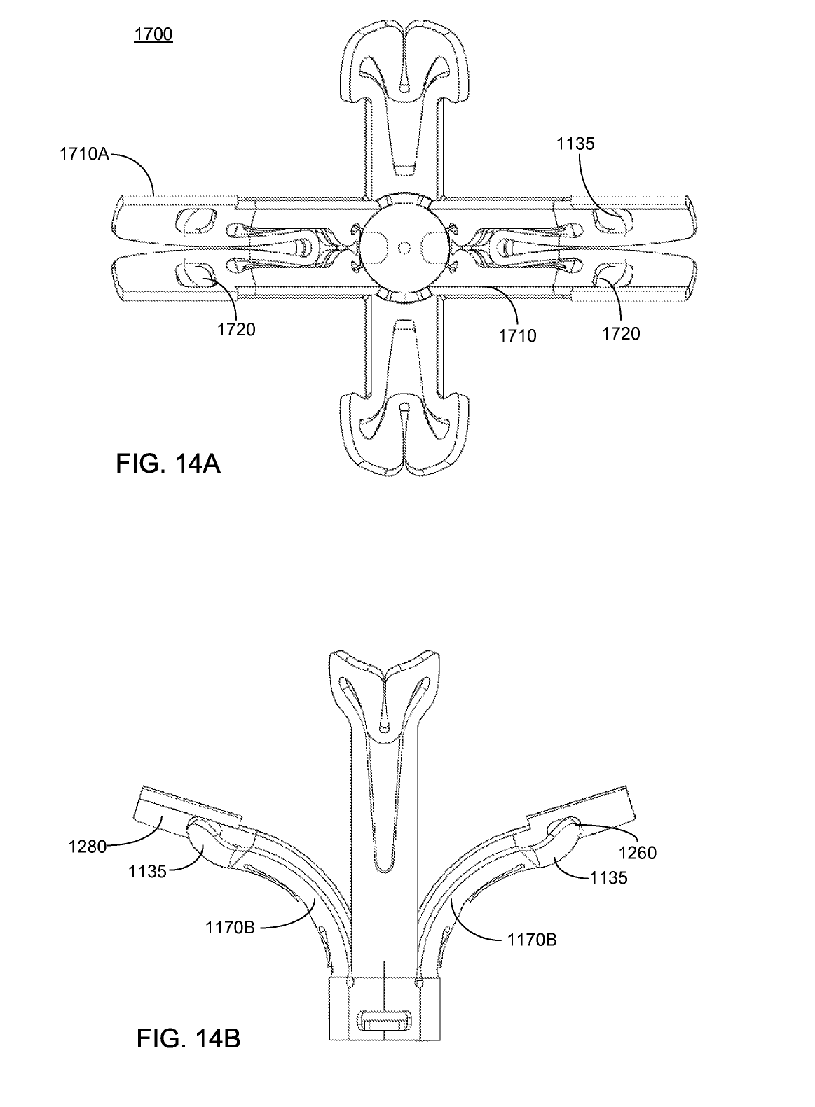

FIG. 13A illustrates a high level side view of a portion of an implant 1600 for repair of a spinal inter-vertebral disc in a deployed configuration; FIG. 13B illustrates a high level schematic view of implant 1600 inserted in a spine; and FIG. 13C illustrates a high level flow chart of the operation of implant 1600, FIGS. 13A-13C being described together. In stage 3000, implant 1600 is provided. Implant 1600 is in all respects similar to implant 1500 of FIGS. 12A-12B, with the exception that a lateral inter-annulus support member 1610 is further provided. In one illustrated embodiment, layer 1510C of one of inter-annulus support members 1510 is replaced with the provided lateral inter-annulus support member 1610. In another embodiment (not shown), lateral inter-annulus support member 1610 is provided in addition to the layers of inter-annulus support members 1510. Inter-annulus support members 1510 are illustrated as comprising three layers, however this is not meant to be limiting in any way and any number of layers may be provided, as described above. Implant 1600 is illustrated as comprising inter-annulus support members 1510 which as described above exhibit layers with different lengths, however this is not meant to be limiting in any way. In another embodiment, implant 1600 comprises any of inter-annulus support members 30, 920 and 1130 described above with the addition of lateral inter-annulus support member 1610. In one embodiment, lateral inter-annulus support member 1610 comprises Nitinol. In one embodiment, later inter-annulus support member 1610 comprises a shape memory alloy. In one further embodiment, lateral inter-annulus support member 1610 comprises a shape memory polymer.

Lateral inter-annulus support member 1610 comprises a pair of arms 1620, a pair of link members 1630, an inter-annulus inner connecting member 1150, an inter-annulus outer connecting member 1160 and further exhibits: a first end 1640; a second end 1650, opposing first end 1640; a first face 1660; and a second face 1670 opposing first face 1660.

First end 1640 of lateral inter-annulus support member 1610 is consonant with outer surface 1190, and in particular is in a line with outer surface 1190, is parallel to longitudinal axis 1180 and is coupled to axial support member 1220. Each arm 1620 is connected to second 1240 of axial support member 1220 by a respective link member 1630. As described above, inter-annulus inner connecting member 1150 and inter-annulus outer connecting member 1160 are arranged to connect link members 1630 and provide support thereto. Preferably, arms 1620 are arranged to be positioned in close proximity to each other such that nucleus pulposus cannot exit there between. In one embodiment, arms 1620 are arranged to mate with each other. Arms 1620 are constructed and arranged such that the surface of first face 1660 is wide enough to cover a tear 280 of annulus 270 in a target mammal.

In one embodiment, first face 1660 of each link member 1630 concavingly curves away from second end 1240 of axial support member 1220 into the respective arm 1620 and first face 1660 of each arm 1620 convexingly curves away from the respective link member 1630 into second end 1650 of lateral inter-annulus support member 1610, i.e. first face 1660 of lateral inter-annulus support member 1610 is generally tilde shaped. In one embodiment, first face 1660 of each link member 1630 generally proceeds from second end 1240 of axial support member 1220 along longitudinal axis 1180 into the concave curve. In another embodiment first face 1660 of each link member 1630 generally proceeds along a separate axis into the concave curve, the separate axis exhibiting an acute angle with longitudinal axis 1180, as described above in relation to axis 1183 of FIG. 9D. Second face 1670 of lateral inter-annulus support member 1610 generally faces axial support member 1220. In one embodiment, second face 1660 of each link member 1630 convexingly curves away from second end 1240 of axial support member 1220 into the respective arm 1620 and second face 1660 of each arm 1620 convexingly curves away from the respective link member 1630 into second end 1650 of lateral inter-annulus support member 1610.

The extension of each arm 1620 into second end 1650 extends along a plane 1680. In one embodiment, the angle between plane 1680 and longitudinal axis 1180, is 0°-45°. As will be described below, when deployed, first face 1660 of lateral inter-annulus support member 1610 is arranged to meet inner wall 271 of annulus 270. Advantageously, because of the angle between plane 1680 and longitudinal axis 1180, second end 1650 of lateral inter-annulus support member 1610 does not meet inner wall 271 of annulus 270, thereby when lateral inter-annulus support member 1610 presses against inner wall 271 pressure is applied by the curved, or flat, portion of arms 1620 and not the edge of second end 1650, the edge of which could damage inner wall 271 of annulus 270. In one embodiment (not shown), lateral inter-annulus support member 1610 comprises a plurality of layers, as described above in relation to inter-annulus support members 1130 of FIGS. 9A-9F.

As illustrated in FIG. 13B, annulus 270 exhibits tear 280 in the posterior wall of annulus 270. In stage 3010, a channel 1690 is surgically created through the lateral wall of annulus 270 as part of a discectomy and implant 1600 is inserted through channel 1690 into area 275 of annulus 270, as described above in FIGS. 9E-9F in relation to the insertion of implant 1100 into annulus 270. In one embodiment, implant 1600 is delivered into channel 1690 in a delivery position, wherein inter-annulus securing members 1510, lateral inter-annulus securing member 1610 and proximal securing members 1170 are secured so as to not extend past plane 1191 defined by outer surface 1190 of generally tubular member 1120. In another embodiment, proximal securing members 1170 extend past the plane defined by the outer surface of generally tubular member 1120 while in the delivery position. In stage 3020, inter-annulus support members 1510, lateral inter-annulus support member 1610 and proximal securing members 1170 move to a deployed configuration. In stage 3030, each layer of each inter-annulus support member 1510 extends radially outward into a generally concavingly shaped form whose end portion defines a plane exhibiting an angle with a longitudinal axis extending through the center of generally tubular member 1120, as described above in relation to stage 2040 of FIG. 11. In one embodiment, each inter-annulus support member extends along an axis into the generally concavingly shaped form, the axis exhibiting an acute angle with the longitudinal axis of the generally tubular member, the angle being defined from the longitudinal axis of the generally tubular member to the face of the inter-annulus support member facing away from axial support member 1220.

As described above in relation to implant 1100, inter-annulus support members 1510 are arranged to press against inner wall 271, thereby maintaining implant 1600 within annulus 270. Advantageously, inter-annulus support members 1510 press against the lateral section of annulus 270 which is more robust than the posterior section of annulus 270. Additionally, in the flexion position pressure is applied in a direction generally perpendicular to longitudinal axis 1180 and thus does not push implant 1600 out through tear 280.

In stage 3040, lateral inter-annulus support member 1610 is arranged to extend to the posterior wall of annulus 270 and cover tear 280. As described above, lateral inter-annulus support member 1610 is arranged to extend radially, preferably into a generally tilde shape. As described above, lateral inter-annulus support member 1610 is constructed so as to cover tear 280, thereby not allowing nucleus pulposus to exit there through. Ejection force 290 pushing in the direction of tear 280, i.e. in a direction generally perpendicular to longitudinal axis 1180, applies pressure to lateral inter-annulus support member 1610. Advantageously, the concavingly curved shape of first face 1660 of link members 1630 increases the resistance to ejection force 290 and reduces the chance of buckling, as described above in relation to inter-annulus support members 30 of FIGS. 1A-1E and inter-annulus support members 1130 of FIGS. 9A-9F.

FIG. 14A illustrates a high level top view of an implant 1700 for repair of a spinal inter-vertebral disc; and FIG. 14B illustrates a high level side view of implant 1700, FIGS. 14A and 14B being described together. Implant 1700 is in all respects similar to implant 1100 of FIGS. 9A-9F, with the exception that the plurality of inter-annulus support members 1130 are replaced with a plurality of inter-annulus support members 1710 each exhibiting a plurality of layers 1710A and 1710B. Inter-annulus support members 1710 are in all respects similar to inter-annulus support members 1130 of FIGS. 9A-9F, with the exception that each layer 1710A exhibits a pair of holes 1720 arranged to receive arms 1135 of the respective layer 1710B at second end 1260 thereof. Only two layers of each inter-annulus support member 1710 are illustrated, however this is not meant to be limiting in any way and any number of layers may be provided, each layer exhibiting a pair of holes arranged to receive arms 1135 of an adjacent layer, without exceeding the scope. Proximal securing members 1170 are each illustrated as comprising a single layer, however this is not meant to be limiting in any way and any number of layers may be provided without exceeding the scope.

In operation, as described above, pressure is applied to inter-annulus support members 1710 when positioned within an annulus. As each layer 1710A is pressed against the inner wall of the annulus, layer 1710B slides along second face 1280 of the respective layer 1710A until being stopped by arms 1135 entering the respective holes 1720. The layers of each inter-annulus support member 1710 then cooperate to act as a single layer.

The deflection of a beam is given as:

δ=(F*L³)/(3*E*I) EQ. 2

where δ is the deflection of a beam exhibiting a length L, F is the force being applied to the beam, E is Young's Modulus and I is the moment of inertia of the beam. The moment of inertia of a rectangular cross section is given as:

I=(b*h³)/12 EQ. 3

where b is the length of the rectangle, length b being perpendicular to the direction of the applied force F, and h is the height of the rectangle, h being defined in the direction of the applied force F. As can be seen from EQs. 2 and 3, as more layers are added to inter-annulus support member 1710, thus increasing h, the deflection of inter-annulus support member 1710 decreases by a cubic function of the added thickness of inter-annulus support member 1710.

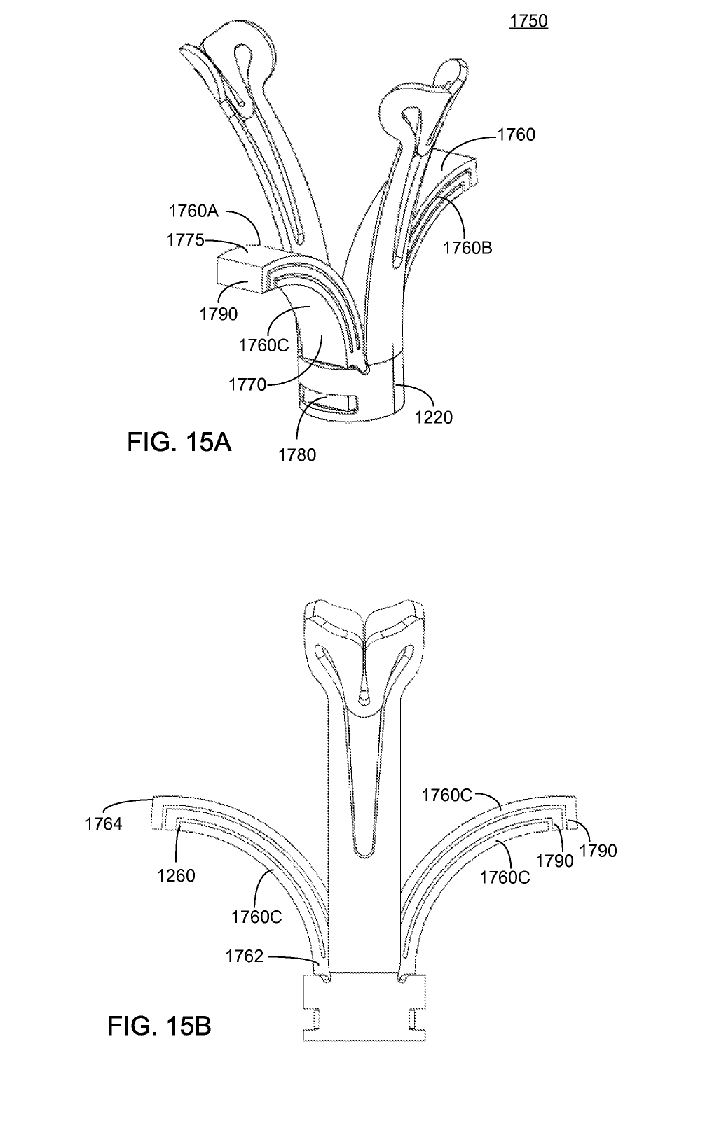

FIG. 15A illustrates a high level perspective view of an implant 1750 for repair of a spinal inter-vertebral disc in a deployed configuration; and FIG. 15B illustrates a high level side view of implant 1750, FIGS. 15A and 15B being described together. Implant 1750 is in all respects similar to implant 1100 of FIGS. 9A-9F, with the exception that the plurality of inter-annulus support members 1130 are replaced with a plurality of inter-annulus support members 1760 each exhibiting a plurality of layers 1760A, 1760B and 1760C. Each layer of inter-annulus support members 1760 exhibits: a first end 1762; a second end 1764, opposing first end 1762; a first face 1770; and a second face 1775, opposing first face 1770. Each layer 1760A and 1760B further exhibit a protrusion 1790 extending from second end 1764 and arranged to block the advance of an adjacent layer when second end 1764 comes in contact with protrusion 1790.

As described above in relation to inter-annulus support members 1130, first face 1770 of layer 1760A faces second face 1775 of layer 1760B, first face 1770 of layer 1760A faces second face 1775 of layer 1760C and first face 1770 of layer 1760C generally faces axial support member 1220. Three layers of each inter-annulus support member 1760 are illustrated, however this is not meant to be limiting in any way and any number of layers may be provided without exceeding the scope. First ends 1762 of each layer of a particular inter-annulus support member 1760 are connected to axial support member 1220, preferably as a unitary block 1780. As described above in relation to inter-annulus support members 1130, first face 1770 of each layer of inter-annulus support members 1760 extends in a concave curve to second end 1764 thereof and second face 1775 extends in a convex curve to second end 1764 thereof. Proximal securing members 1170 are each illustrated as comprising a single layer, however this is not meant to be limiting in any way and any number of layer may be provided without exceeding the scope.

As described above in relation to implant 1700, when pressure is applied to inter-annulus support members 1760 the advance of each layer is blocked by the protrusion 1770 of the adjacent layer and the layers cooperate together to act as a single layer. Therefore, as described above, the deflection of each inter-annulus support member 1760 decreases by a cubic function of the added thickness of inter-annulus support member 1760.

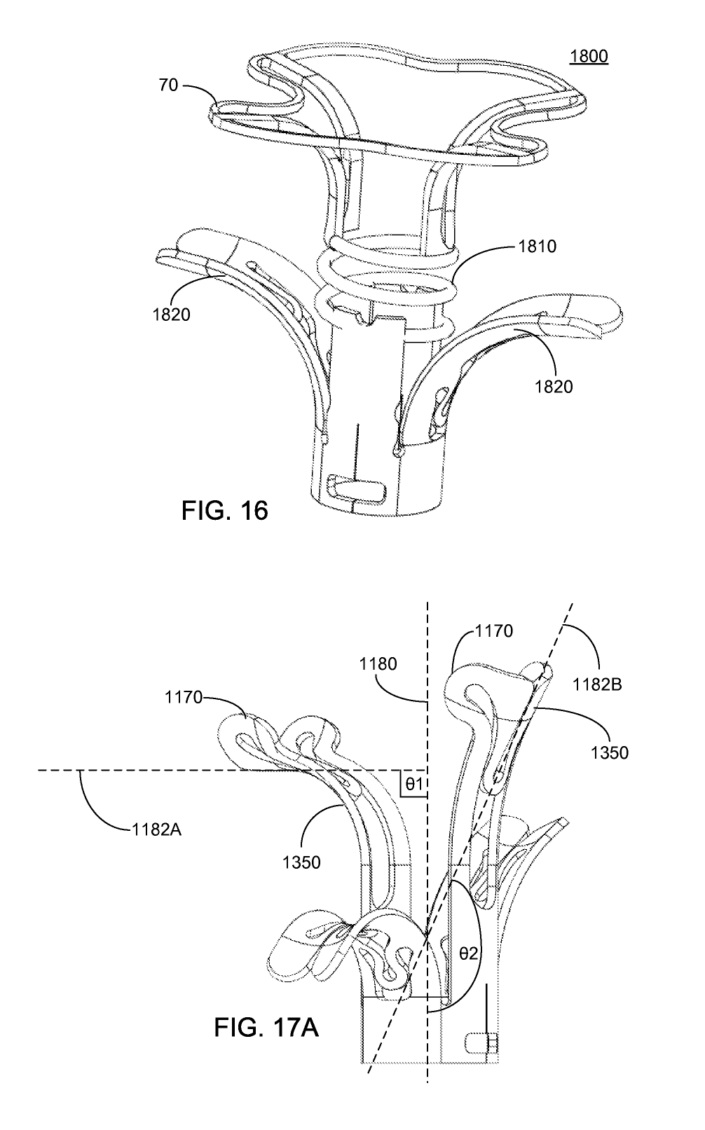

FIG. 16 illustrates a high level perspective view of an implant 1800 for repair of a spinal inter-vertebral disc in a deployed configuration. Implant 1800 is in all respects similar to implant 10 of FIGS. 1A-1E with the exception that proximal extenders 240 are replaced with a spring 1810. Additionally, in one illustrated embodiment, inter-annulus support members 30 are replaced by inter-annulus support members 1820 which are in all respects similar to any of the layers of inter-annulus support members 1130 of FIGS. 9A-9F. In another embodiment, implant 1800 is provided with a plurality of inter-annulus support members 1130 as described above in relation to FIGS. 9A-9F. In another embodiment, implant 1800 is provided with a plurality of support members 30 as described above in relation to FIGS. 1A-1E.

In operation, as described above in relation to FIG. 1D, in one embodiment proximal securing members 70 are situated external of the annulus. Therefore, spring 1810 allows positioning of implant 1800 within the annulus while maintaining proximal securing members 70 external of the annulus, regardless of the thickness of the channel through which implant 1800 is inserted.

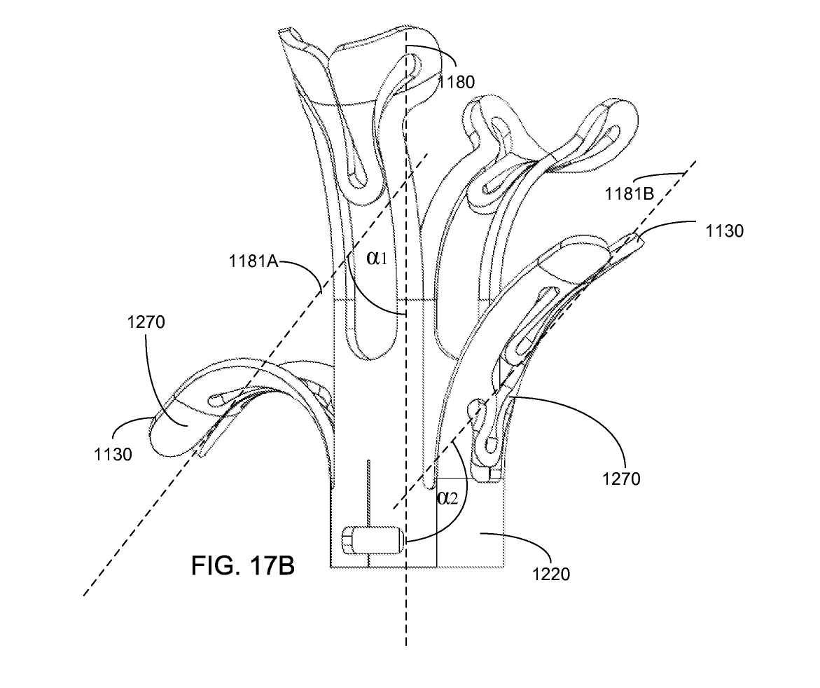

FIG. 17A illustrates a first high level side view of a portion of implant 1100; and FIG. 17B illustrates a second high level side view of the portion of implant 1100, FIGS. 17A and 17B being described together. For ease of understanding, only a single layer of inter-annulus support members 1130 and proximal securing members 1170 are illustrated. As described above, when implant 1100 is situated within an annulus, pressure is applied to inter-annulus support members 1130 and proximal securing members 1170. In one embodiment, each inter-annulus support member 1130 and each proximal securing member 1170 can bend independently of each other into different angles. In particular, as described above in relation to FIG. 9A, each inter-annulus support member 1130 extends generally to define a plane 1181 at an end thereof, plane 1181 exhibiting an angle α with longitudinal axis 1180, angle α defined from first face 1270 to longitudinal axis 1180. In one embodiment, angle α is 45°-120° thereby each inter-annulus support member 1130 generally forms an arc of a circle exhibiting a corresponding central angle of 60°-135°. Each inter-annulus support member 1130 extends into a unique plane 1181, the planes denote 1181A and 1181B. Each plane 1181 exhibits a unique angle α with longitudinal axis 1180 thereby allowing adaptation to the uneven shape of the walls of the annulus, the angles denoted α1 and α2. In one embodiment, each proximal securing member 1170 extends into a unique plane 1182, the planes denote 1182A and 1182B. Each plane 1182 exhibits a unique angle θ with longitudinal axis 1180 thereby allowing adaptation to the uneven shape of the walls of the annulus, the angles denoted θ1 and θ2 and defined from first face 1350 to longitudinal axis 1180. In one embodiment, angle θ is 90°-150° and thereby each proximal securing member 1170 generally forms an arc of a circle exhibiting a corresponding central angle of 30°-90°.

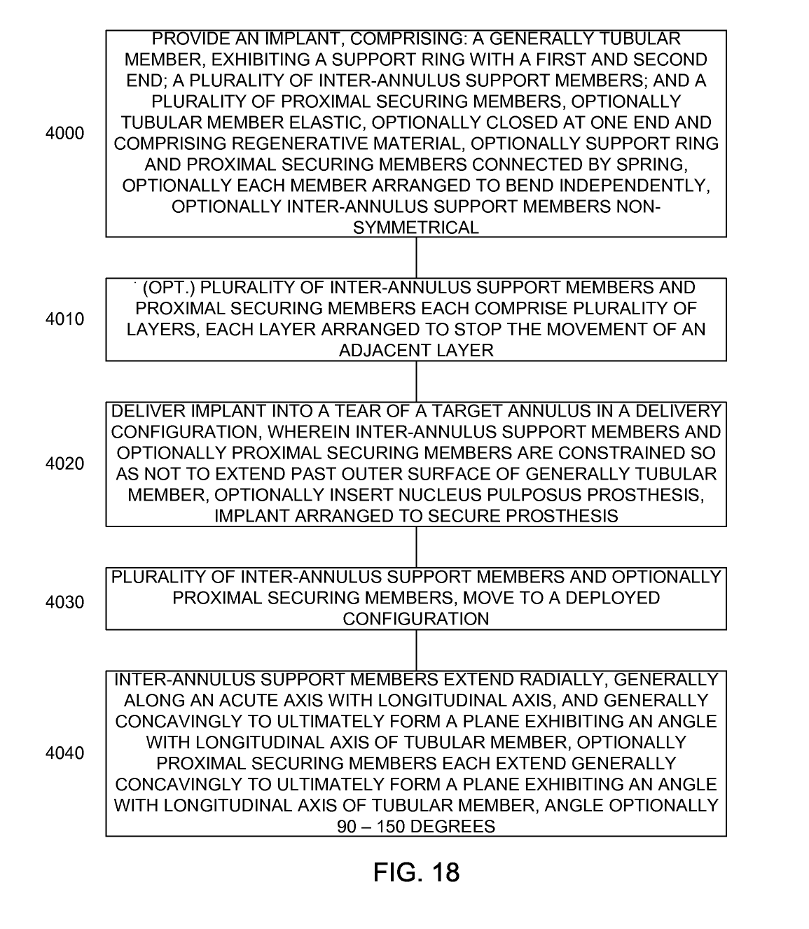

FIG. 18 illustrates a high level flow chart of a second method for repairing a spinal inter-vertebral disc. In stage 4000, an implant is provided, such as one of implants 1500, 1700, 1750 and 1800. The implant comprises: a generally tubular member, exhibiting a axial support member with a first end and a second end opposing the first end; a plurality of inter-annulus support members, such as inter-annulus support members 1130, 1510, 1710, 1760 and 1820; and a plurality of proximal securing members, such as proximal securing members 70 or 1170.

Optionally, the generally tubular member, the plurality of inter-annulus support members and the plurality of proximal securing members are formed of a unitary tube, thus ensuring maximum resistance to ejection forces, as described above. In one embodiment, the generally tubular member is elastic thereby allowing positioning into a target annulus regardless of the shape thereof. In one embodiment, the generally tubular member is closed at one end by a flow blocking member, such as flow blocking member 1175, thereby forming a reservoir. The reservoir is in one embodiment filled with regenerative material such as stem cells and growth factors, without limitation. In one embodiment, the axial support member and the plurality of proximal securing members are connected by a spring, such as spring 1810 of implant 1800.

In one embodiment, each inter-annulus support member and proximal securing member are arranged to bend independently of each other to different angles. In one embodiment, the inter-annulus support members are non-symmetrical, i.e. exhibit different lengths, as described above in relation to inter-annulus support members 1510 of FIGS. 12A-12B.

In optional stage 4010, the plurality of inter-annulus support members of stage 4000 each comprise a plurality of layers, as described above in relation to implant 1100. Optionally, the plurality of proximal securing members of stage 4000 each comprise a plurality of layers, as described above in relation to implant 1100. In one embodiment, each layer of each inter-annulus support member is arranged to stop the movement of an adjacent layer there along, as described above in relation to implants 1700 and 1750.

In stage 4020, the implant of stage 4000 and/or optional stage 4010 is delivered into a tear in a target annulus in a delivery position, wherein the plurality of inter-annulus support members, and the plurality of proximal securing members, are secured so as to not extend past the plane defined by the outer surface of the generally tubular member. In another embodiment, the plurality of proximal securing members are arranged to extend past the plane defined by the outer surface of the generally tubular member while in the delivery configuration. In one embodiment, a nucleus pulposus prosthesis is inserted into the intervertebral disc of the target annulus, the implant delivered into the target annulus is arranged to secure the inserted nucleus pulposus prosthesis. In one embodiment, the nucleus replacement treatment comprises replacing nucleus pulposus with any one, or a combination, of: a mechanical nucleus; a polymer based material, pre-formed or in situ formed, such as a hydrogel, a thermo-responsive polymer, silicon, polymerized water in oil emulsion and PMMA (Polymethyl methacrylate), without limitation; and a tissue engineered material using a synthetic or natural scaffold, without limitation. In stage 4030, the plurality of inter-annulus support members of stage 4000, and optionally the plurality of proximal securing members of stage 4000, move to a deployed configuration, as described above.

In stage 4040, each of the plurality of inter-annulus support members of stage 4000, and/or of optional stage 4010, in the deployed configuration of stage 4030, extends radially outward, in one embodiment into a generally concavingly shaped form whose end portion defines a plane exhibiting an angle with a longitudinal axis extending through the center of the generally tubular member of stage 4000. Specifically, the angle is defined from the face of each inter-annulus support member facing the axial support member of the generally tubular member. As described above, in one embodiment the angle is 45°-120°. In one embodiment, the angle is less than, or equal to, 90°, optionally 45°-90°. In one embodiment, each inter-annulus support member extends along an axis into the generally concavingly shaped form, the axis exhibiting an acute angle with the longitudinal axis of the generally tubular member, the angle being defined from the longitudinal axis of the generally tubular member to the face of the inter-annulus support member not facing the axial support member. In one embodiment, each of the plurality of inter-annulus support members of stage 4000 comprises a pair of link members, each coupled to the second end of the axial support member. Optionally, the pair of link members exhibit between each other an angle of 45°-90°, further optionally an angle of about 80°.

In one embodiment, each proximal securing member extends into a generally concavingly shaped form whose end portion defines a plane exhibiting an angle with a longitudinal axis extending through the center of the generally tubular member of stage 4000. Specifically, the angle is defined from the face of each proximal securing member facing the axial support member of the generally tubular member. As described above, in one embodiment, the angle is 90°-150°, optionally each proximal securing member arranged to curve into a plane exhibiting a unique angle with the longitudinal axis of the generally tubular member.

In one embodiment, the various embodiments of implants described above are implemented of a biodegradable material, thus exiting the patient body after a predetermined period.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

1-59. (canceled)

60. An implant for repair of a spinal inter-vertebral disc, the implant comprising:
an axial support member exhibiting a longitudinal axis, an outer surface, a distal end and a proximal end;
at least one inter-annulus support member having a first end, a second end opposing the first end, a first link member, a first face and a second face opposing the first face, the first link member arranged to connect the first end of said at least one inter-annulus support member to said axial support member, said at least one inter-annulus support member having a deployed configuration wherein the second end of said at least one inter-annulus support member extends away from the longitudinal axis of said axial support member, the first face of the first link member generally facing the axial support member in the deployed configuration and generally concavingly curved, and the second face of the first link member generally convexingly curved, wherein the first face and second face of the first link member generally proceed proximally from said axial support member along an extension axis into the curve, the extension axis exhibiting an acute angle with the longitudinal axis, the acute angle defined from the second face of the first link member to the longitudinal axis; and
at least one proximal securing member positioned proximal of said at least one inter-annulus support member, said at least one proximal securing member having a first end and a second end opposing the first end, the first end of said at least one proximal securing member connected to said axial support member.

61. The implant of claim 60, wherein:
said at least one proximal securing member comprises a plurality of proximal securing members arranged radially about said axial support member; and
said at least one inter-annulus support member comprises a plurality of inter-annulus support members arranged radially about said axial support member.

62. The implant according to claim 60, wherein said at least one proximal securing member is displaced from said at least one inter-annulus support member along the longitudinal axis of said axial support member by the thickness of a target annulus, such that the second face of said at least one inter-annulus support members meets the inner wall of the target annulus and said at least one proximal securing member meets the outer wall of the target annulus.

63. The implant according to claim 60, wherein said at least one proximal securing member is displaced from said at least one inter-annulus support member along the longitudinal axis of said axial support member by less than the thickness of a target annulus, such that the second face of said at least one inter-annulus support member meets the inner wall of the target annulus and said at least one proximal securing member meets the medial portion of the target annulus.

64. The implant according to claim 60, wherein said at least one proximal securing member comprises a plurality of layers.

65. The implant according to claim 60, wherein the second end of said at least one inter-annulus support member is wider than the first end of said at least one inter-annulus support member.

66. The implant according to claim 60, further comprising a barrier material arranged to block the flow of nucleus pulposus from an annulus of the spinal inter-vertebral disc to external of said axial support member.

67. The implant according to claim 60, further comprising a first flow blocking member having a first end and a second end opposing the first end, the first end of said first flow blocking member connected to said axial support member, the second end of said first flow blocking member extending towards the longitudinal axis of said axial support member when said at least one inter-annulus support member is in the deployed configuration.

68. The implant according to claim 60, wherein each of said inter-annulus support members comprises a plurality of layers, each layer arranged to meet an adjacent layer of said inter-annulus support member responsive to a distal force applied to said inter-annulus support member.

69. The implant according to claim 60, wherein said at least one inter-annulus support member, in said deployed configuration, extends into a plane, said plane exhibiting an angle with the longitudinal axis of said axial support member of greater than 90 degrees, wherein said angle is defined between said first face of said at least one inter-annulus support member and the longitudinal axis of said axial support member.

70. The implant according to claim 60, further comprising:
a proximal securing member positioned proximal of said at least one inter-annulus support member, said at least one proximal securing member having a first end and a second end opposing the first end, the first end of said at least one proximal securing member connected to said axial support member,
wherein said at least one inter-annulus support member generally extends along a support member axis and said proximal securing member generally extends along a securing member axis, said support member axis rotated about said longitudinal axis of said axial support member in relation to said securing member axis.

71. The implant according to claim 60, further comprising a lateral inter-annulus support member having:
a first end;
a second end opposing the first end;
a second link member;
an arm;
a first face; and
a second face opposing the first face,
wherein the second link member is arranged to connect the first end of said lateral inter-annulus support member to said axial support member,
wherein said lateral inter-annulus support member has a delivery configuration wherein said lateral inter-annulus support member does not extend past a plane defined by the outer surface of said axial support member, and a deployed configuration wherein the second end of said lateral inter-annulus support member extends away from the longitudinal axis of said axial support member past the plane defined by the outer surface of said axial support member,
wherein the first face of the second link member generally faces the axial support member in the deployed configuration and generally convexingly curves from the second end of the axial support member and the second face of the second link member generally concavingly curves from the second end of the axial support member, the first face and second face of the second link member generally proceeding along an extension axis exhibiting an acute angle with the longitudinal axis into the curve, the acute angle being defined from the first face of the second link member,
wherein the convex curve of the first face of the second link member extends into the first face of the arm, the first face of the arm generally concavingly curved from the first face of the second link member, and
wherein the concave curve of the second face of the second link member extends into the second face of the arm, the second face of the arm generally convexingly curved from the second face of the second link member.

72. The implant according to claim 60, further comprising a second flow blocking member connected to the distal end of said axial support member, said second flow blocking member arranged to prevent the extrusion of regenerative material contained within said axial support member.

73. The implant according to claim 60, wherein said at least one inter-annulus support member comprises a plurality of layers, each layer arranged in said deployed configuration to arrest movement of an adjacent layer, at a predetermined point, caused by force applied to the adjacent layer.

74. The implant according to claim 73, wherein each layer of said at least one inter-annulus support member exhibits a hole arranged to receive an end of an adjacent layer.

75. The implant according to claim 73, wherein each layer of said at least one inter-annulus support member exhibits a protrusion arranged, in said deployed configuration, to come in contact with an end of an adjacent layer, thereby arresting movement of the adjacent layer, at a predetermined point, caused by force applied to the adjacent layer.

76. A method for repairing a spinal inter-vertebral disc, the method comprising:
providing an implant comprising:
an axial support member exhibiting a longitudinal axis, an outer surface, a distal end and a proximal end; and
at least one inter-annulus support member having a first end, a second end opposing the first end, a first link member, a first face and a second face opposing the first face, the first link member arranged to connect the first end of said at least one inter-annulus support member to said axial support member, said at least one inter-annulus support member having a deployed configuration wherein the second end of said at least one plurality of inter-annulus support members extends away from the longitudinal axis of said axial support member, the first face of the first link member generally facing the axial support member in the deployed configuration and generally concavingly curved, and the second face of the first link member generally convexingly curved, wherein the first face and second face of the first link member generally proceed proximally from said axial support member along an extension axis into the curve, exhibiting an acute angle with the longitudinal axis, the acute angle defined from the second face of the first link member to the longitudinal axis;
delivering said provided implant into a target annulus; and
moving said at least one inter-annulus support member into the deployed configuration,
wherein in the deployed configuration said at least one inter-annulus support member is arranged to come in contact with an inner wall of the target annulus.

77. The method according to claim 76, further comprising:
providing a flow blocking member connected to the distal end of said axial support member; and
depositing regenerative material within said axial support member,
wherein said provided flow blocking member is arranged to prevent the extrusion of said deposited regenerative material from said axial support member.

78. The method according to claim 76, further comprising:
providing a lateral inter-annulus support member having:
a first end;
a second end opposing the first end;
a second link member;
an arm;
a first face; and
a second face opposing the first face,
wherein the second link member is arranged to connect the first end of said lateral inter-annulus support member to said axial support member,
wherein said lateral inter-annulus support member has a delivery configuration wherein said lateral inter-annulus support member does not extend past a plane defined by the outer surface of said axial support member, and a deployed configuration wherein the second end of said lateral inter-annulus support member extends away from the longitudinal axis of said axial support member past the plane defined by the outer surface of said axial support member,
wherein the first face of the second link member generally faces the axial support member in the deployed configuration and generally convexingly curves from the second end of the axial support member and the second face of the second link member generally concavingly curves from the second end of the axial support member, the first face and second face of the second link member generally proceeding along an extension axis exhibiting an acute angle with the longitudinal axis into the curve, the acute angle being defined from the first face of the second link member,
wherein the convex curve of the first face of the second link member extends into the first face of the arm, the first face of the arm generally concavingly curved from the first face of the second link member, and
wherein the concave curve of the second face of the second link member extends into the second face of the arm, the second face of the arm generally convexingly curved from the second face of the second link member; and
moving said provided lateral inter-annulus support member into the deployed configuration such that said provided lateral inter-annulus support member is juxtaposed with a tear in the posterior wall of the target annulus,
wherein said delivering is through a lateral wall of the target annulus.

79. The method according to claim 76, further comprising:
inserting a nucleus pulposus prosthesis into the intervertebral disc of the target annulus,
wherein said delivered implant is arranged to secure said inserted nucleus pulposus prosthesis.
